(19) United States
(12) Patent Application Publication
(10) Pub. No.: US 2004/0248765 A1
(43) Pub. Date: Dec. 9, 2004

(54) Tumour targeting prodrugs activated by metallo matrixproteinases

(76) Inventors: David John Mincher, East Lothian (GB); Agnes Turnbull, Edinburgh (GB); Michael Charles Bibby, Bingley (GB); Paul Michael Loadman, Bradford (GB)

Correspondence Address: FINNEGAN, HENDERSON, FARABOW, GARRETT & DUNNER, LLP, 1300 I STREET, NW, WASHINGTON, DC, 20005, US

(21) Appl. No.: 10/469,956

(22) PCT Filed: Mar. 8, 2002
(86) PCT No.: PCT/GB02/01069

(30) Foreign Application Priority Data
Mar. 9, 2001 (GB) 0105929.4

(51) Int. Cl. A61K 38/17; C07K 14/00

(52) U.S. Cl. 514/2; 530/409

(57) ABSTRACT

A-(B)ₙ—X (I)

A-(B)ₙ-Q (II)

### Structural Relationship of Example (8) to its Potential Metabolites

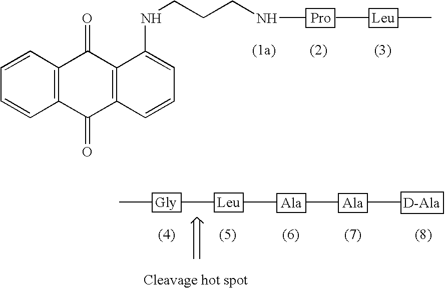

Numbers in Parentheses denote examples in the specification

A compound is of general formula (I) wherein (A) is a moiety comprising one of more of a heterocylcic ring, a carbocyclic ring and a fused ring system, the ring or ring system being essential for a biological activity of the compound by action at a nucleic acid or protein target (B) is a bivalent spacer molecule attached directly to the ring system n is an integer 0 or 1 and (X) is a monovalent moiety containing a amide bond that is cleavable by the action of a matrix metalloproteinase enzyme such as to produce a compound of formula (II) wherein the efficacy of the biological activity of the compound of formula (II) is increased over that of the compound of formula (I).

The present invention relates to compounds that are activated in vivo from relatively biologically inactive compounds, so called ‘prodrugs’, to relatively biologically active ‘drug’ compounds. The invention particularly provides compounds that are inactivated because of their inclusion of a peptide or proteinaceous moiety which results in suppression of their action at a biological target site, completely or in part, which are susceptible to cleavage of all or part of that moiety from the remainder of the compound by one or more metalloproteinase enzymes (MMPs), such as to render the compound more active at said biologically active site.

The present invention particularly provides compounds which incorporate said MMP cleavable peptide or proteinaceous moiety which, when activated by said cleavage, act directly upon a protein or nucleic acid, particularly a receptor or DNA target, such as to effect a desired physiological change. Examples of such change include cell death, whether through apoptosis, lysis or other intermediate effects, and receptor mediated effects, such as glucocorticoid activity, e.g. an anti-inflammatory response.

The ability of cancer cells to metastasise to distant sites in the body is responsible for the majority of cancer-related deaths, either directly due to the spread of metastases to critical organs of the body, or indirectly due to the therapy which is trying to control the metastatic spread. Current chemotherapeutic approaches to the treatment of cancer are limited by the normal tissue toxicity of the majority of agents, leading to a low therapeutic index (Double and Bibby, 1989). There is still a clear need for new tumour-specific chemotherapeutic agents with a high therapeutic index.

The matrix metalloproteinases (MMPs) are a group of zinc atom-dependent endopeptidases which appear to play a major role in the metastatic process (Stetler-Stevenson et al, 1996, Kleiner and Stetler-Stevenson, 1999, Westermarck and Kahari, 1999). There are currently at least 16 members of the MMP family that have been cloned and sequenced (Kleiner and Stetler-Stevenson, 1999) and these can be divided into at least four sub groups based on substrate specificities: the interstitial collagenases, the stromelysins, the gelatinases and more recently the membrane type MMPs (MT-MMPs). The role of these MMPs is to degrade the basement membrane which consists mainly of collagen (MacDougall and Matrisian 1995). These proteolytic enzymes are also linked to many normal processes such as angiogenesis and chronic inflammatory and other degenerative diseases. The MMPs are secreted from the tumour cell in their inactive latent forms (proforms) with activation following cleavage of the prodomain by a range of other proteases such as serine proteases and urokinase-type plasminogen activator (Birkedal-Hansen et al 1993, Kleiner and Stetler-Stevenson, 1993). MMP activity is also controlled by a group of endogenous inhibitors, the tissue inhibitors of metalloproteinases (TIMPs). It is the ratio of MMP activity to TIMP expression which is crucial in the metastatic process. Low TIMP expression correlates with the metastatic potential of murine and human tumour cell lines (Ponton et al 1991, Tsuchiya et al 1993)

One current approach to controlling metastatic disease is to use synthetic inhibitors to inhibit the activity of these proteinases. Two such inhibitors using the MMPs as a therapeutic target are batimastat (Wang et al 1994, Rasmussen and McCann, 1997) and marimastat (Steward, 1999), which are currently under clinical investigation. They have both been shown to inhibit the metastatic spread of tumours in experimental models.

There are many detailed studies in which substrates have been developed to detect the presence of MMP activity. These peptidyl substrates (of 4, 5 or 6 amino acids long) contain a quenched fluorogenic group at the end of the sequence (Nagase 1994, Bickett et al 1993, Bickett et al 1994) which is liberated following proteolytic activity. The sequence of amino acids in these substrates is obviously crucial (Soyez et al 1996).

MMP-2 and MMP-9, which degrade type IV collagen, have been shown to be over-expressed in many malignant tissues including colon (Pyke et al 1993), breast (Monteagudo et al 1990), NSCLC (Urbanski et al 1992, Brown et al 1993) and other tumour types (Sato et al 1992). This overexpression has been detected using techniques such as immunohistochemistry and in situ hybridisation and been correlated with the invasive nature of the tumours (Stetler-Stevenson et al, 1996).

It is essential for the development of a prodrug approach that increased levels of the activating enzymes are restricted to the inimediate tumour environment, otherwise normal tissue toxicity would result. In situ hybridisation studies suggest that mRNA expression of many of the MMPs are localised in the stromal cells of the tumour (MacDougall and Matrisian 1995). Several groups have studies plasma/serum levels of MMPs as markers of malignant activity (Iizasa et al 1999, Nakajima et al 1993, Endo et al 1997). Though levels are often reported raised in cancer patients the immunological assays used do not distinguish between the proform (latent—92 kDa for MMP-9 and 72 kDa for MMP-2) and the active form (84 kDa for MMP-9 and 67 kDa for MMP-2). It is difficult to envisage a situation where patients have high levels of active proteolytic enzymes in the plasma or serum.

It is known, e.g. from U.S. Pat. No. 6,080,575, to provide pre- or pro-proteins or nucleic acid vectors encoding therefor, that are inactive until a part thereof is cleaved by tissue specific enzymes to yield ‘mature’ or otherwise physiologically active forms of proteins. It is also known, e.g. from WO 00/59930, WO 99/44628 and WO 97/14416 (all Merck & Co., Inc.), to conjugate biologically active non-peptide or proteinaceous compounds to a prostate specific antigen cleavable moiety to modulate its activity. The oligopeptide typically is negatively charged at physiological pH, such that the charge inhibits its entry into cells without cleavage of that moiety from the compound.

A specific example of the latter patented conjugate is further described in Nature Medicine, 6, November 2000 which describes a conjugate of doxorubicin with the peptide N-glutaryl-(4-hydroxyprolyl)ala-ser-cyclohexylglycyl-gln-ser-leu-COOH, that peptide being covalently linked between the aminoglycoside of doxorubicin and the C-terminus of the peptide. PSA mediated cleavage of the conjugate, presumably at the ‘gln-ser’ bond if the cleavage is directly analogous to that of the parental peptide (the specificity of cleavage is not reported in the paper), gives principally the known cytotoxic agents ‘leu-dox’ and ‘dox’.

The conjugate is reported to be non-cytotoxic (IC₅₀s>100 μM) compared to ‘leu-dox’ and ‘dox’. The mechanism by which peptide conjugation diminishes cytotoxic activity is not discussed, but it can be seen that the peptide has a negatively charged carboxylate group at physiological pH which would confer reduced capacity for cellular uptake.

It is already known that, for certain series of cytotoxic anthraquinone peptide conjugate compounds, selection of C-terminally free ‘gly-gly’, ‘gly-ala’ and ‘gly-phe’ conjugates of formula

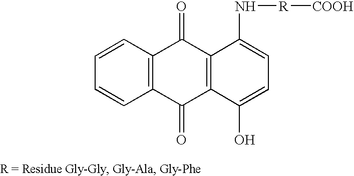

gave a complete lack of in vitro cytotoxicity. This is demonstrated to be explainable by a lack of cellular uptake (see I. Meikle et al. Anti Cancer Drug Design, 1995, 10, 515) since the negative charge may prevent uptake of drug, which will most likely proceed by passive diffusion as for many of the established anti-tumour drugs (B. C. Baguley, Anti Cancer Drug Design, 1991, 6, 1). In contrast their hydrophobic, C-terminally esterified derivatives are actively taken up by cells and are actively cytotoxic.

A preferred cleavage substrate for MMP-9 has been reported by McGeehan et al (1994). McGeehan et al (1994) started with a parent substrate: Dnp-pro-leu-gly˜leu-trp-ala-(D)arg-NH₂ a fluorogenic substrate reported earlier (Stack and Gray, 1989), and utilised 88 unique amino acid substitutions at each position; over the 4 subsites (P₂ through P₂′) 352 potential substrates were evaluated. This study, combined with results of earlier work (Berman et al, 1992), gave an extended profile of the substrate specificities of both MMP-1 and MMP-9 with emphasis on MMP-1.

The present inventors now provide compounds which are activated from a relatively biologically inactive state to a relatively biologically active state on cleavage of, or within, a peptide or proteinaceous moiety forming part of said compounds by action of a matrix metalloproteinase enzyme. Particularly preferred compounds of the present invention may enter cells, but cannot interact effectively with a target, such as DNA or a receptor therein or thereon, without cleavage of all or part of the peptide or proteinaceous moiety.

Thus in a first aspect of the present invention there is provided a compound of general formula (I)

A-(B)ₙ—X (I)

wherein A is a moiety comprising one of more of a heterocyclic ring, a carbocyclic ring and a fused ring system, the ring or ring system being essential for a biological activity of the compound by action at a nucleic acid or protein target

B is a bivalent spacer molecule attached directly to the ring system

n is an integer 0 or 1

and X is a monovalent moiety containing a amide bond that is cleavable by the action of a matrix metalloproteinase enzyme such as to produce a compound of formula II

A-(B)ₙ-Q (II)

wherein the efficacy of the biological activity of the compound of formula II is increased over that of the compound of formula (I).

In preferred forms the ring is not a phenyl ring directly attached to the α-carbon of an amino acid residue, more preferably not any ring being so directly attached.

The amide bond is preferably a peptide bond between amino acids in a peptide chain X which is cleaved to result in a shorter peptide chain Q. Alternatively the bond is an amide bond between two moieties Xa and Xb, one or both of which include peptide analogues, such as isosteres where the peptide bonds between amino acids are replaced by other bonds of different chemical nature.

By “isosteres” of the amino acids or peptides we include ω-amino acids that have side chains that mimic the characteristic side chains of α-amino acid and peptides used in the invention. Examples of conventional isosteres are illustrated in ‘A Practical Guide to Combinatorial Chemistry, (1997) Edits. Czamik and DeWitt, American Chemical Society ISBN 0-8412-3485-X, page 57, FIG. 2, e.g. depsipeptides and peptoids, wherein the sidechains characteristic of α-amino acids are in alternative carried on ester group carbons or on amide group nitrogens; and in Medicinal Chemistry: Principles and Practice (1998) Edit: F D King, The Royal Society of Chemistry, ISBN-0-85186-494-5, Chapter 14, see Tables 2 page 208 re carboxylic amide groups in peptides; both incorporated herein by reference Also included are peptide mimics corresponding to peptides with amide bonds replaced by olefinic bonds.

More preferably the first aspect provides a compound of general formula (III)

A-(B)ₙ—(Xaa)ₘY (III)

wherein A is a moiety comprising one or more of a heterocyclic ring, a carbocyclic ring, and a fused ring system, the ring or ring system being essential for therapeutic activity of the compound by action at a nucleic acid or protein target

B is a bivalent spacer moiety

n is an integer 0 or 1

Xaa is any amino acid residue

m is an integer from 2 to 100 and

Y is H, a cation or a capping group

wherein Xaa is independently selected at each repeat occurrence such as to form an oligopeptide or protein which is internally cleavable by a Matrix Metalloproteinase enzyme such as to produce a compound of formula (IV)

A-(B)ₙ—(Xaa)_{q}-Y (IV)

wherein A, B, n, Xaa and Y have the meanings ascribed in formula III and q is 0 or an integer less than m.

such that the efficacy of compound of formula IV at the nucleic acid or protein is increased over the efficacy of the compound of formula III.

Preferably the activity is one of modulation of the activity of a cell membrane or intracellular receptor, an enzyme, or modulation of the ability of DNA to be replicated or transcribed or expressed.

Preferably the compound of formula IV has higher efficacy at the nucleic acid or protein target than a compound of formula V

A-(B)ₙ-Y (V)

wherein A, B, n and Y are as described above.

Moiety A is preferably that comprising only one of a fused ring system, which may be heterocyclic or carbocyclic, or is an unfused heterocyclic ring. Non-limiting examples of moiety A include steroid ring systems, anthracene ring systems and mitomycin ring systems, which are capable of interacting with protein receptors or nucleic acid. More specific ring systems are those that are found in known biologically active molecules daunorubicin,, mitoxanthrone, methotrexate, camnptothecin, quinocarmycin, chlorambucil and mitomycin-C. A is conveniently linked to the spacer B or directly to X or (Xaa)ₘ through a nitrogen, such as in an amino group attached directly or indirectly to the ring.

For example, where the ring system is that of the anthracenes dox- or daunorubicin, B, X or Xaa may be attached to the primary amino group of the amino sugar, daunosamine. In mitoxantrone B, X or Xaa may be attached via amide bonds with the secondary amino group in that system. With steroids B, X or Xaa may be conjugated to one or more of the 3, 17 or 7 positions via ether or ester linkages to the steroid hydroxyl groups.

Moiety B may be any bivalent spacer that is consistent with the compound of formula II having activity at the intended target. Typical spacers are alkylene, α-ω-diamino, α-ω-dicarboxylate, α-ω-dialcohol, α-ω-aminoalcohol, α-ω-amino acid moieties. Other suitable α-ω substituted alkyl group combinations will occur to those skilled in the art. Preferred spacer groups will be of 2 to 10 contiguous atoms in length between A and the amino acid residue of (Xaa)ₘ to which B is linked. More preferably this is 4, 5 or 6 atoms long, e.g. —NH(CH₂)n-C(O)—, —NH(CH₂)n-NH—, —NH(CH₂)n-O—, wherein n is 2, 3 or 4, e.g. —NH(CH₂)₂—C(O)—, —NH(CH₂)₃—NH—, —NH(CH₂)₄—O—.

The oligopeptide chain (Xaa)ₙ is preferably of 4 to 30 amino acid residues long, most preferably of 4 to 15 and still more preferably 4 to 10 amino acid residues long, while the moiety (Xaa)_{q} is 1 to 8, more preferably 1 to 4 amino acid residues long.

Preferred identities for (Xaa)ₘ comprise an oligopeptide chain of formula (VI), also herein given as SEQ ID No 1:

-Xaa1-Xaa2-Xaa3-Xaa4-

wherein Xaa1-4 are amino acid residues wherein

Xaa1 is that of proline or a non-naturally occurring analogue thereof.

Xaa2 is that of leucine, a non-naturally occurring analogue thereof, S-mercaptoethyl cysteine (EMC), thienylalanine (THA) and p-chlorophenylalanine (PFC)

Xaa3 is that of glycine, a non-naturally occurring analogue thereof, be thienylalanine (THA) or cyclohexylalanine (CHA) and

Xaa4 is that of leucine, a non-naturally occurring analogue thereof, S-methylcysteine (SMC), norvaline (NVA), norleucine (NLE) or phenylglycine (PHG).

It will be evident to those skilled in the art that whilst amino acids may be of L-form, they may also advantageously be selected in the D- form, such as that of D-proline and D-leucine. The D-isomer will provide some improved resistance to degradation of its bond to spacer B or to Xaa2 by peptidases.

Still more preferably the oligopeptide chain -(Xaa)ₘ is of formula (VII) or SEQ ID No 2:

-Xaa1-Xaa2-Xaa3-Xaa4-Xaa5-

wherein Xaa1-4 are residues as described above and Xaa5 is the residue of any amino acid. Preferably residue Xaa5 is selected from L-or D-amino acids tyrosine, methionine, phenylglycine, isoleucine, leucine and norvaline, but more preferably is a tyrosine residue or a non-naturally occurring analogue thereof.

Still more preferably the oligopeptide chain -(Xaa)ₘ- is of formula (VIII) or SEQ ID No3:

-Xaa1-Xaa2-Xaa3-Xaa4-Xaa5-Xaa6-

wherein Xaa1-5 are residues as described above and Xaa6 the residue of any amino acid, but more preferably is of D or L-alanine or a non-naturally occurring analogue thereof.

Still more preferably the oligopeptide chain -(Xaa)ₘ- is of formula (IX) or SEQ ID No 4:

-Xaa1-Xaa2-Xaa3-Xaa4-Xaa5-Xaa6-Xaa7-

wherein Xaa1-6 are residues as described above and Xaa7 is the residue of L- or D-alanine, lysine or ornithine (ORN). Advantageously Xaa7 is a D-amino acid residue

The capping group Y may be H, OH, a cation or any group conventionally used to cap a peptide chain in pharmaceutical use. Examples of such a group are described or exemplified in WO 95/09149, WO 99/65886 and WO 01/44190 all of which are incorporated herein by reference. Preferred compounds of the invention have formula where A is any ring as described in these incorporated patents or any spacer group as described in these incorporated patents for the cytotoxic active is compounds of their inventions.

A preferred class of compound of the invention is acted upon by one or more specific matrix metalloproteinases, particularly by MMP-2 or MMP-9, most preferably by MMP9, such as to increase its cytotoxicity to tumour cells.

Conveniently such compound, termed prodrug, is based upon an anthraquinone ring system linked to be peptide through a bivalent spacer group such as that described in WO 95/09149, WO 99/65886 or WO 01/44190, all of applicant BTG International Ltd.

These are of general formula

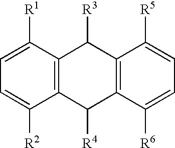

wherein R¹ and R² are independently hydrogen or hydroxyl, R³ and R⁴ are independently oxo or hydrogen (the single bond shown being double in the case of oxo), one of R⁵ and R⁶ is —B-(Xaa)ₚ-Y, where p is any integer and may be m or q as hereinbefore described, and the other is hydrogen, hydroxyl or a group B, wherein the or each B is independently a spacer group providing —NH—, —C(O)— or —O— in the bond with (Xaa)ₚ, if present, at least one group B does not provide the residue of an α-amino acid adjacent the anthraquinone nucleus and the B of any B-(Xaa)ₚ moiety is joined to the anthraquinone nucleus via an —NH— bond. These compounds are described as being particularly useful antitumour compounds acting via topoisomerases and also to be useful as dyes.

For the purpose of the present invention the peptide to which A or A-B— is conjugated is an oligopeptide of three to ten amino acid residues long. Such a compound is activated to a compound of formula II having activity as a topoisomerase inhibitor which is cytotoxic to tumour cells expressing topoisomerase I, IIα or IIβ. More preferably the oligopeptide is from five to eight amino acid residues long and a most convenient form is a heptapeptide sequence. The oligopeptide sequence may be linked in turn to a further non-peptide group or groups at one or more positions other than the point of conjugation to the ‘drug’ moiety.

In this manner it is possible to generate a prodrug which is converted to an active and potent compound within the tumour environment by overexpressed matrix metalloproteinases. This allows far greater tumour/normal tissue ratios than are currently achievable with existing topoisomerase inhibitor chemotherapeutics and therefore increases the therapeutic index.

In contrast to prior art prodrugs the diminished cytotoxicity of prodrugs of the present invention does not depend on this general and non-specific mechanism of deactivation through inhibition of entry into cells. Preferred compounds are both N-terminally cationic and hydrophobic permitting active cellular uptake and lack of cytotoxicity depends upon specific deactivation of e.g. DNA-directed mechanism of cell kill. In the case of the preferred anthraquinone compounds there is diminished DNA-binding and interaction with topoisomerase enzymes.

In a second aspect of the present invention there is provided a pharmaceutical preparation comprising a pharmaceutically acceptable carrier and/or excipient and a compound of the first aspect. Any suitable pharmaceutically acceptable carrier can be used. The preparation should be suitable for administration in the chosen manner. In particular, it should be sterile and, if intended for injection, non-pyrogenic.

Administration of the aforementioned compounds of the invention or a formulation thereof need not be restricted by route. Options include enteral (for example oral and rectal) or parenteral (for example delivery into the nose or lung or injection into the veins, arteries, brain, spine, bladder, peritoneum, muscles or subcutaneous region). The compounds may be injected directly into the tumour. The treatment may consist of a single dose or a plurality of doses over a period of time. The dosage will preferably be determined by the physician but may be between 0.01 mg and 1.0 g/kg/day, for example between 0.1 and 500 mg/kg/day. In terms of dose per square meter of body surface, the compound can be administered at 1.0 mg to 1.5 g per m² per day, for example 3.0-200.0 mg/m²/day. At least some compounds of the invention have a particularly low toxicity to normal mammalian cells and could be given in quite high doses, for example 50-300 mg/kg. By comparison doxorubicin has a maximum tolerated dose of 5 mg/kg in rodents and 1-2 mg/kg in man.

Whilst it is possible for a compound of the invention to be administered alone, it is preferable to present it as a pharmaceutical formulation, together with one or more acceptable carriers and/or excipients. The carrier(s) and/or excipients must be “acceptable” in the sense of being compatible with the compound of the invention and not deleterious to the recipients thereof.

The formulations may conveniently be presented in unit dosage form and may be prepared by any of the methods well known in the art of pharmacy. A unit dosage form may comprise 2.0 mg to 2.0 g, for example 5.0 mg to 300.0 mg of active ingredient. Such methods include the step of bringing into association the active ingredient, i.e. the compound of the invention, with the carrier and/or excipients which constitute one or more accessory ingredients. In general the formulations are prepared by uniformly and intimately bringing into association the active ingredient with liquid carriers or finely divided solid carriers and/or excipients and/or two or all of these, and then, if necessary, shaping the product.

Formulations in accordance with the present invention suitable for oral administration may be presented as discrete units such as capsules, cachets or tablets, each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or granules, optionally mixed with a binder (e.g. povidone, gelatin, hydroxypropyl-methyl cellulose), lubricant, inert diluent, preservative, disintegrant (e.g. sodium starch glycollate, PVP, cross-linked povidone, cross-linked sodium carboxymethyl cellulose), surface-active or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethylcellulose in varying proportions to provide desired release profile.

Formulations suitable for topical administration in the mouth include lozenges comprising the active ingredient in a flavoured basis, usually sucrose and acacia or tragacanth; pastilles comprising the active ingredient in an inert basis such as gelatin and glycerin, or sucrose and acacia; and mouth-washes comprising the active ingredient in a suitable liquid carrier.

Formulations suitable for parenteral administration include aqueous and non-aqueous sterile injection solutions which may contain anti-oxidants, buffers, bacteriostats and solutes which may render the formulation isotonic with the blood of the intended recipient; and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

Preferred unit dosage formulations are those containing a daily dose or unit, daily sub-dose or an appropriate fraction thereof, of an active ingredient.

It should be understood that in addition to the ingredients particularly mentioned above the formulations of this invention may include other agents conventional in the art having regard to the type of formulation in question, for example those suitable for oral administration may include flavouring agents.

At least some of the compounds of the present invention are useful as anticancer, antiviral and/or antiparasitic drugs and at least some of the anticancer compounds can be used against most malignancies.

Particular tumours suitable for treatment in accordance with the invention include cancers of the uterine cervix, head, neck, brain gliomas, breast, colon, lung, prostate, skin, mouth, nose, oesophagus, stomach, liver, pancreas and metastatic forms of any of these which express matrix metalloproteinases.

Particular viral infections suitable for treatment in accordance with the invention include those caused by the viruses herpes simplex virus I (HSV I); herpes simplex virus II (HSV II); varicella-zoster virus/Ellen (VZV Ellen); bovine papilloma virus (BPV); and human immunodeficiency virus (HIV).

Particular protozoal infections suitable for treatment in accordance with the invention include trichomoniasis; malaria (especially that caused by Plasmodium falciparum); trypanosomiasis (caused by Trypanosoma brucei and T. cruzi); and leishmaniasis. It will be appreciated by those skilled in the art that the novel profile of activity of the present compounds will make it likely that some at least will be useful as antibacterial agents.

In a third aspect of the present invention there is provided a method of treating a human or animal body in need of therapy for a disorder wherein the tissue affected by the disorder produces matrix metalloproteinase (MMP), particularly MMP-9, and particularly at an increased level as compared to other tissues, comprising administering to said human or animal body an effective therapeutic dose of a compound or preparation of the invention. Preferred disorders are selected from the group consisting of cancer, viral infection or parasitic infection.

In a fourth aspect of the present invention there is provide a compound of the first aspect of the invention for use in therapy.

In a fifth aspect of the present invention there is provided the use of a compound of the first aspect of the invention for the manufacture of a medicament for the treatment of a disease state associated with tissue expressing a matrix metalloproteinase, particularly a cancer.

The preferred anthracene ring compounds of the invention, which are prodrug anthraquinones acting as topoisomerase inhibitors, are investigated below and clearly exhibit the MMP-9 mediated selective cleavage of the scissile peptide bonds, e.g. gly-leu, in vitro and in living cells. Furthermore, the dual properties of lipophilicity and cationic charge associated with such preferred compounds may circumvent p170 (p-glycoprotein) mediated multi drug resistance (T. J. Lampidis et al. Biochemistry, 1997, 36, 2679, Circumvention of pgp MDR as a function of anthracycline lipophilicity and charge).

A sixth aspect of the present invention provides the use of an oligopeptide of comprising a sequence of general formula (IV) or SEQ ID No 1

-Xaa1-Xaa2-Xaa3-Xaa4-

as an activity modulator for incorporation by conjugation into a biologically active molecule having action at a protein or nucleic acid target, the incorporation being such that the biologically active molecule is made more active in the presence of matrix metalloproteinase than when it is absent.

The present invention will now be described further by reference to the following non-limiting examples and Figures. These are provided for the purpose of illustration only and other examples falling within the scope of the claims will occur to those skilled in the art in the light of these.

### FIGURES

FIG. 1 shows the structural relationship of one of the preferred compounds of the invention, Example 8, to its potential in vivo metabolites. Numbers in parenthesis denote examples in the specification.

FIG. 2 is an HPLC chromatogram of the separation of Example 8 (NU:UB187) and its potential degradation products.

FIG. 3 is a Mass Spectrum of metabolites generated following incubation of Example 8 [NU:UB 187] with human recombinant MMP-9 and parent ion spectrum of metabolites.

FIG. 4 shows products of action of tumour homogenates of HT1080 on NU:UB227

FIGS. 5 to 22 show structures of the compounds of the Examples.

FIG. 23 shows a scheme for synthesis of Example 8.

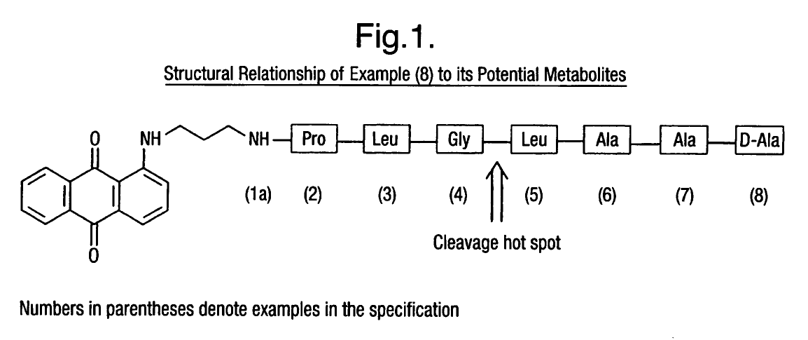

### GENERAL METHODS

### Synthesis of Anthraquinone Spacer Compounds

### Method A: General Method for the Preparation of Mono-Aminated Anthraquinone-Spacer Arm Compounds from Amines

The appropriate (unsubstituted or substituted) monochloroanthraquinone (5 mmol) was suspended in DMSO (15 cm³); an appropriate α,ω-diaminoalkane (50 mmol) was added and the mixture was heated for 1 h over a boiling water bath (or heated at reflux as appropriate). The solution was cooled and added to a large excess of water (500 cm³). The red precipitated solid was filtered off, dried and could be used for subsequent reactions without further purification. Analytically pure samples were obtained by column chromatographic purification on silica gel 60[Merck] using chloroform methanol and gradient elution.

note. In examples in which the monochloroanthraquinone additionally contained hydroxyl groups, pyridine was used in place of DMSO.

### Method B: General Method for the Preparation of mono-aminated-4-hydroxylated-anthraquinone-spacer Arm Compounds from Amines

1,4-Dihydroxyanthraquinone (1 mmol) was suspended in ethanol (20 cm³) and THF (20 cm³) containing an appropriate α,ω-diaminoalkane (10 mmol) and heated over a water bath (at 95° C.) for 1.75 h. The solution was cooled and di-tert-butyl-dicarbonate (20 mmol) in methanol (40 cm³) was added dropwise and the reaction mixture was allowed to reach room temperature. The crude N-^{t}Boc protected compound was extracted into chloroform and applied to a silica gel chromatography column, using toluene:ethyl acetate (4:1) as the eluting solvent, to give the N-^{t}Boc protected compound which was deprotected using trifluoroacetic acid to give the 4-hydroxylated-anthraquinone-spacer arm compound as the water soluble trifluoroacetate salt.

### Method C: General Method for the Preparation of mono-aminated-4,8-dihydroxylated-anthraquinone-spacer Arm Compounds

Leuco-1,4,5-trihydroxyanthraquinone (3 mmol) was suspended in dichloromethane (200 cm³). An N-^{t}Boc-α,ω-diaminoalkane or α,ω-diaminoalkane^{#} (3 mmol) was added and the mixture was stirred at room temperature for 6 h followed by the addition of triethylamine (2 cm³) and aeration for 2 h. The mixture was purified by silica gel chromatography using a dichloromethane:ethyl acetate gradient to give the spacer compound in its N-^{t}Boc protected form which was deprotected using trifluoroacetic acid.

^{#} NOTE: Where the appropriate amine was not available in its mono-N-^{t}Boc protected form di-tert-butyl dicarbonate (3 mmol) in methanol (100 cm³) was added to the reaction mixture after aeration.

### Method D: General Method for the Activation of an N-α-Protected Amino Acid by Conversion to a Pentafluorophenolate Ester

Pentafluorophenol (1.1 mmol) was added to a stirred solution of an N-protected amino acid (1 mmol) in dry ethyl acetate (40 cm³) at 0° C. A solution of dicyclohexylcarbodiimide (1.2 mmol) in dry ethyl acetate (10 cm³) was added dropwise and stirring was continued for 12 h as the mixture was allowed to reach room temperature. The precipitated dicyclohexylurea was filtered off and the solution evaporated to yield a crystalline precipitate of the N-protected amino acid-O-pentafluorophenolate ester which was used for subsequent reaction without further purification.

### Method E: General Method for Coupling of an N-^{t}Boc (tert-butoxycarbonyl) Protected-C-Activated Amino Acid to an aminoalkylamino-anthraquinone or to an anthraquinone-amino Acid/Peptide Conjugate and Subsequent Deprotection of the Reaction Product

The anthraquinone conjugate (1 mmol) was suspended in DMF [and triethylamine (1 mmol) to liberate the free amine where the anthraquinone conjugate is a trifluoroacetate salt] and stirred at 0° C. An N-^{t}Boc protected amino acid-O-pentafluorophenolate ester (1.1 mmol) in DMF [or an N-^{t}Boc protected amino acid-N-hydroxysuccinimide ester (1.1 mmol) in THF] was added drop-wise and the reaction mixture was allowed to reach room temperature. Stirring was continued for a further 12 h. The mixture was partitioned between chloroform and water. The chloroform extracts were washed with saturated sodium bicarbonate_{(aq)}, then water, dried (MgSO₄), filtered, evaporated to a low volume and purified by silica gel column chromatography eluting initially with chloroform: ethyl acetate (4:1). The major product was eluted using the same solvent system containing increasing gradients of methanol (0.5-2% v/v). Fractions containing the major product were combined, filtered and evaporated.

The ^{t}Boc protected compound was dissolved in trifluoroacetic acid at room temperature. After 0.5 h the solvent was evaporated and the residual solid re-evaporated with ethanol (3×10 cm³) before dissolving in a minimum volume of ethanol (3 cm³). Addition of ether (100 cm³) gave a precipitate of the deprotected anthraquinone spacer-linked amino acid conjugate as the N-terminal trifluoroacetate salt which was filtered off and dried in vacuo.

### Method F: General Method for Coupling of an N-Fmoc (9-fluorenylmethoxy-carbonyl) Protected-C-Activated Amino Acid to an aminoalkylamino-anthraquinone or to an anthraquinone-amino Acid/Peptide Conjugate and Subsequent Deprotection of the Reaction Product

The anthraquinone conjugate (1 mmol) was suspended in DMF [and triethylamine (1 mmol) to liberate the free amine where the anthraquinone conjugate is a trifluoroacetate salt] and stirred at 0° C. An N-Fmoc protected amino acid-O-pentafluorophenolate ester (1.1 mmol) in DMF [or an N-Fmoc protected amino acid-N-hydroxysuccinimide ester (1.1 mmol) in THF] was added drop-wise and the reaction mixture was allowed to reach room temperature. Stirring was continued for a further 12 h. The mixture was purified by solvent extraction and silica gel column chromatography as described in Method E.

The Fmoc protected compound was dissolved in 20% (v/v) piperidine in DMF (20 cm³) and stirred at room temperature for 5 min. The solution was partitioned between chloroform and water (1:1, 100 cm³), washed with water (3×50 cm³), dried (Na₂SO₄), filtered and evaporated to a low volume before application to a silica gel chromatography column [chloroform:methanol (19:1)] eluting with chloroform:methanol, (increasing gradient, 19:1→5:1). The fractions containing the product were combined, evaporated to dryness and dissolved in trifluoroacetic acid. After 5 minutes the solvent was evaporated and dissolved in a minimum volume of ethanol (3 cm³). Addition of ether (100 cm³) gave a precipitate of the deprotected anthraquinone spacer-linked amino acid conjugate as the N-terminal trifluoroacetate salt.

### EXAMPLES

### Example (1a)

### 1-[(3-Aminopropyl)amino]anthraquinone.[Method A]

Prepared using 1-chloroanthraquinone and 1,3-diaminopropane. Mp 106-108° C.

### Example (1b)

### 1-[(3-Aminopropyl)amino]anthraquinone trifluoroacetate

Compound (1a) was purified by column chromatography, eluting with butanol:glacial acetic acid:water (4:5:1). 1-[(3-aminopropyl)amino]anthraquinone acetate was partitioned between chloroform and water and neutralised with triethylamine to give 1-[(3-aminopropyl)amino]anthraquinone. The chloroform extract was dried (Na₂SO₄), filtered, evaporated to dryness and dissolved in a small volume of tri-fluoroacetic acid (2 cm³). Addition of ether (100 cm³) afforded the title compound as a red solid. Mp 196° C. ESMS(+)(Cone 20V) m/z: 281 (100%)(RNH₃)⁺, 263 (20%), 102 (15%).

### Example (2)

### 1-[3-(L-Propylamino)propylamino]anthraquinone trifluoroacetate.[Method E]

Prepared from the reaction of 1-[(3-aminopropyl)amino]anthraquinone (1a) with N-^{t}Boc-L-proline-N-hydroxysuccinimide ester in THF. Mp 176° C. ¹H nmr spectrum (300 Mz, d₆-DMSO) δ: 1.80-2.05 (4H, m); 2.30 (2H, m); 3.10-3.60 (6H, m); 4.10 (1H, t); 7.25 (1H, d); 7.45 (1H, d); 7.65 (1H, t); 7.80-7.95 (2H, m); 8.10-8.20 (2H, m); 8.70 (1H, t); 9.00 (2H, br s); 9.70 (1H, t). FABMS(+) m/z 378 (12%)(RNH₂)⁺, 176 (31%), 89 (51%), 77 (80%), 31 (100%). M, 491.

### Example (3)

### 1-[3-(L-Leucyl-L-prolylamino)propylamino]anthraquinone trifluoroacetate. [Method E]

Prepared from the reaction of N-^{t}Boc-L-leucine-N-hydroxysuccinimide ester and 1-[3-(L-prolylamino)propylamino]anthraquinone trifluoroacetate (2) in THF and triethylamine. Mp 126° C. ESMS(+)(Cone 20V) m/z: 513 (4%)(RNH₂+Na)⁺, 491 (100%)(RNH₃)⁺, 119 (10%), 87 (30%), 55 (2%). ESMS(−)(Cone 50V) m/z: 113 (70%)(CF₃COO)⁻, 69 (100%). M, 604.

### Example (4)

### 1-[3-(Glycyl-L-leucyl-L-prolylamino)propylamino]anthraquinone trifluoroacetate. [Method E]

Prepared by the reaction of N-^{t}Boc-glycine-N-hydroxysuccinimide ester with 1-[3-(L-leucyl-L-prolylamino)propylamino]anthraquinone trifluoroacetate (3) in TBF and triethylamine. Mp 144-148° C. ESMS(+)(Cone 20V) m/z: 570 (15%)(RNH₂+Na)⁺, 548 (100%)(RNH₃)⁺, 239 (10%), 87 (75%), 55 (5%). M, 661.

### Example (5)

### 1-[3-(L-Leucyl-glycyl-L-leucyl-L-prolylamino)propylamino]-anthraquinone trifluoroacetate. [Methods D and E]

N-^{t}Boc-L-leucylglycine was converted to its pentafluorophenolate ester and reacted with 1-[3-(L-leucyl-L-prolylamino)propylamino]anthraquinone trifluoroacetate (3) in DMF and triethylamine. Mp 144° C. ESMS(+)(Cone 50V) m/z: 683 (10%)(RNH₂+Na)⁺, 661 (50%)(RNH₃)⁺, 491 (2%), 378 (100%), 155 (5%), 87 (35%). M, 774.

### Example (6)

### 1-[3-(L-Alanyl-L-leucyl-glycyl-L-leucyl-L-prolylamino)propylamino]-anthraquinone trifluoroacetate. [Method E]

N-^{t}Boc-L-alanine-N-hydroxysuccinimide ester was reacted with 1-[3-(L-leucyl-glycyl-L-leucyl-L-prolyl-amino)propylamino]anthraquinone trifluoroacetate (5) in THF and triethylamine. Mp 168° C. ESMS(+)(Cone 20V) m/z: 754 (5%)(RNH₂+Na)⁺, 732 (45%)(RNH₃)⁺, 239 (15%), 119 (15%), 87 (100%). M, 846.

### Example (7)

### 1-[3-(L-Alanyl-L-alanyl-L-leucyl-glycyl-L-leucyl-L-prolylamino)-propylamino]anthraquinone trifluoroacetate. [Methods D and E]

N-^{t}Boc-L-alanyl-L-alanine was converted to its pentafluorophenolate ester and reacted with 1-[3-(L-Leucyl-glycyl-L-leucyl-L-prolylamino)propylamino]anthraquinone trifluoroacetate (5). Mp 132° C. ESMS(+)(Cone 50V) m/z: 825 (12%)(RNH₂+Na)⁺, 803 (25%)(RNH₃)⁺, 378 (75%), 239 (5%), 119 (20%), 87 (100%). ESMS(−)(Cone 50V) m/z: 113 (CF₃COO)⁻, 69 (100%). M, 917.

### Example (8)

### 1-[3-(D-Alanyl-L-alanyl-L-alanyl-L-leucyl-glycyl-L-leucyl-L-prolylamino)-propylamino]anthraquinone trifluoroacetate. [Method E]

Prepared from the reaction of N-^{t}Boc-D-alanine-N-hydroxysuccinimide ester and 1-[3-(L-alanyl-L-alanyl-L-leucyl-glycyl-L-leucyl-L-prolylamino)propylamino]anthraquinone trifluoroacetate (7) in THF and triethylanine. Mp 166° C. ESMS(+)(Cone 50V) m/z: 896 (25%)(RNH₂+Na)⁺, 874 (80%)(RNH₃)⁺, 497 (65%), 378 (100%)(Aq-Spacer-Pro-NH₂)⁺, 239 (10%), 87 (100%). ESMS(−)(Cone 50V) m/z: 113 (100%)(CF₃COO)⁻. M, 988.

### Example (9)

### 1-[3-(D-Prolylamino)propylamino]anthraquinone trifluoroacetate. [Methods D and E]

N-^{t}Boc-D-Proline was converted to its pentafluorophenolate ester and reacted with 1-[(3-aminopropyl)amino]anthraquinone (1a) in DMF and triethylamine. Mp 177° C. ¹H nmr spectrum (300 MHz, d₆-DMSO) δ: 1.70-2.00 (5H, m); 2.20 (1H, m); 3.20-3.50 (6H, m); 4.05 (1H, t); 7.25 (1H, dd); 7.45 (1H, dd); 7.65 (1H, m); 7.80-7.95 (2H, m); 8.05-8.10 (2H, m); 8.60 (1H, t); 9.00 (2H, br. s); 9.75 (1H, t). C₂₄H₂₄N₃O₅F₃ requires: C 58.65, H 4.92, N, 8.55%. Found C 57.84, H 4.76, N 8.35%. FABMS(+) m/z: 400 (4%), 378 (36%)(RNH₂)⁺, 263 ( 10%), 89 (30%), 70 (100%). M, 491.

### Example (10)

### 1-[3-(L-Leucyl-D-prolylamino)propylamino]anthraquinone trifluoroacetate. [Method E]

Prepared from the reaction of N-^{t}Boc-L-leucine-N-hydroxysuccinimide ester and 1-[3-(D-prolylamino)propylamino]anthraquinone trifluoroacetate (9) in TBF and triethylamine. ESMS(+)(Cone 20V) m/z: 491 (100%)(RNH₃)⁺. ESMS(−)(Cone 20V) m/z: 113 (100%)(CF₃COO)⁻. M, 604.

### Example (11)

### 1-[3-(Glycyl-L-leucyl-D-prolylamino)propylamino]anthraquinone trifluoroacetate. [Method E]

Prepared by the reaction of N-^{t}Boc-glycine-N-hydroxysuccinimide ester with 1-[3-(L-leucyl-D-prolylamino)propylamino]anthraquinone trifluoroacetate (10) in THF and triethylamine. ESMS(+)(Cone 20V) m/z: 548 (100%)(RNH₃)⁺. M, 661.

### Example (12)

### 1-[3-(L-Leucyl-glycyl-L-leucyl-D-prolylamino)propylamino]-anthraquinone trifluoroacetate. [Methods D and E]

N-^{t}Boc-L-leucylglycine was converted to its pentafluorophenolate ester and reacted with 1-[3-(L-leucyl-D-prolylamino)propylamino]anthraquinone trifluoroacetate (10) in DMF and triethylamine. ESMS(+)(Cone 20V) m/z: 661 (100%)(RNH₃)⁺. M, 774.

### Example (13)

### 1-[3-(L-Alanyl-L-leucyl-glycyl-L-leucyl-D-prolylamino)propylamino]-anthraquinone trifluoroacetate. [Method E]

N-^{t}Boc-L-alanine-N-hydroxysuccinimide ester was reacted with 1-[3-(L-leucyl-glycyl-L-leucyl-D-prolylamino)propylamino]anthraquinone trifluoroacetate (12) in THF and triethylamine. Mp 168° C. ESMS(+)(Cone 20V) m/z: 732 (100%)(RNH₃)⁺. M, 846.

### Example (14)

### 1-[3-(L-Alanyl-L-alanyl-L-leucyl-glycyl-L-leucyl-D-prolylamino)-propylamino]anthraquinone trifluoroacetate. [Methods D and E]

N-^{t}Boc-L-alanyl-L-alanine was converted to its pentafluorophenolate ester and reacted with 1-[3-(L-leucyl-glycyl-L-leucyl-D-prolylamino)propylamino]anthraquinone trifluoroacetate (12). Mp 132° C. ESMS(+) m/z: 825 (30%)(RNH₂+Na)⁺, 803 (100%)(RNH₃)⁺, 355 (100%), 114 (32%). ESMS(−) m/z: 113 (10%)(CF₃COO)⁻, 69 (35%). M, 917.

### Example (15)

### 1-[3-(D-Alanyl-L-alanyl-L-alanyl-L-leucyl-glycyl-L-leucyl-D-prolylamino)propylamino]anthraquinone trifluoroacetate. [Method E]

Prepared from the reaction of N-^{t}Boc-D-alanine-N-hydroxysuccinimide ester and 1-[3-(L-alanyl-L-leucyl-glycyl-L-leucyl-D-prolylamino)propylamino]anthraquinone trifluoroacetate (14) in THF and triethylamine. Mp 166° C. ESMS(+)(Cone 90V) m/z: 896 (5%), 874 (25%)(RNH₃)⁺, 497 (15%), 378 (100%)(Aq-Spacer-Pro-NH₂)⁺. ESMS(−) (Cone 90V) m/z: 145 (100%), 113 (88%)(CF₃COO)⁻. M, 988.

### Example (16)

### 1-[3-(L-Norvalyl-glycyl-L-leucyl-L-prolylamino)propylamino]-anthraquinone trifluoroacetate. [NU:UB 260] [Methods D and E]

N-^{t}Boc-L-norvaline was converted to its pentafluorophenolate ester and reacted with 1-[3-(glycyl-L-leucyl-L-prolylamino)propylamino]anthraquinone trifluoroacetate (4) in DMF and triethylamine. Mp 210° C. FAB(+) m/z: 670 (50%)(RNH₂+Na)⁺, 648 (100%)(RNH₃)⁺. M, 760.

### Example (17)

### 1-[3-(L-Alanyl-L-norvalyl-glycyl-L-leucyl-L-prolylamino)propylamino]-anthraquinone trifluoroacetate. [NU:UB 261] [Method E]

N-^{t}Boc-L-alanine-N-hydroxysuccinimide ester was reacted with 1-[3-(L-norvalyl-glycyl-L-leucyl-L-prolyl-amino)propylamino]anthraquinone trifluoroacetate (16) in THF and triethylamine. Mp 198° C. FAB(+) m/z: 740 (100%)(RNH₂+Na)⁺, 718 (90%)(RNH₃)⁺. M, 831.

### Example (18)

### 1-[3-(L-Alanyl-L-alanyl-L-norvalyl-glycyl-L-leucyl-L-prolylamino)-propylamino]anthraquinone trifluoroacetate. [NU:UB 262] [Methods E]

N-^{t}Boc-L-alanine-N-hydroxysuccinimide ester was reacted with 1-[3-(L-alanyl-L-norvalyl-glycyl-L-leucyl-L-prolylamino)propylamino]anthraquinone trifluoroacetate (17). Mp 197° C. ESMS(+)(Cone 20V) m/z: 811 (10%)(RNH₂+Na)⁺, 789 (100%)(RNH₃)⁺, 414 (10%), 307 (5%), 129 (55%), 97 (80%). ESMS(−)(Cone 20V) m/z: 113 (CF₃COO)⁻, 69 (100%). M, 902.

### Example (19)

### 1-[3-(D-Alanyl-L-alanyl-L-alanyl-L-norvalyl-glycyl-L-leucyl-L-prolylamino)propylamino]anthrauinone trifluoroacetate [Methods D and E]

Prepared from the reaction of N-^{t}Boc-D-alanine-N-hydroxysuccinimide ester and 1-[3-(L-alanyl-L-alanyl-L-norvalyl-glycyl-L-leucyl-L-prolylamino)propylamino]-anthraquinone trifluoroacetate (18) in THF and triethylamine. Mp 208° C. ESMS(+)(Cone 20V) m/z: 882 (5%)(RNH₂+Na)⁺, 860 (100%)(RNH₃)⁺, 449 (10%), 129 (70%), 97 (100%). ESMS(−)(Cone 20V) m/z: 113 (60%)(CF₃COO)⁻69 (100%). M, 973.

### Example (20)

### 1-[3-(L-Norleucyl-glycyl-L-leucyl-L-prolylamino)propylamino]-anthraquinone trifluoroacetate. [Method F]

Prepared by the reaction of N-Fmoc-L-norleucine pentafluorophenolate ester with 1-[3-(glycyl-L-leucyl-L-prolylamino)propylamino]anthraquinone trifluoroacetate (4) in DMF and triethylamine. Mp 169° C. FABMS(+) m/z: 683 (50%)(RNH₂+Na)⁺, 661 (75%)(RNH₃)⁺, 378 (15%), 182 (10%), 132 (100%). M, 774.

### Example (21)

### 1-[3-(L-Alanyl-L-norleucyl-glycyl-L-leucyl-L-prolylamino)propylamino]anthraquinone trifluoroacetate. [Method E]

Prepared from the reaction of N-^{t}Boc-L-alanine-N-hydroxysuccinimide ester and 1-[3-(L-norleucyl-glycyl-L-prolylamino)propylamino]anthraquinone trifluoroacetate (20) in THF and triethylamine. Mp 185° C. FABMS(+) m/z: 754 (10%)(RNH₂+Na)⁺, 732 (6%)(RNH₃)⁺, 378 (6%), 132 (100%). M, 845.

### Example (22)

### 1-[3-(L-Alanyl-L-alanyl-L-norleucyl-glycyl-L-leucyl-L-prolylamino)-propylamino]anthraquinone trifluoroacetate. [Methods D and E]

N-^{t}Boc-L-alanyl-L-alanine was converted to its pentafluorophenolate ester and reacted with 1-[3-(L-norleucyl-glycyl-L-leucyl-L-prolylamino)propylamino]anthraquinone trifluoroacetate (20) in DMF and triethylamine. Mp 176° C. FABMS(+) m/z: 825 (52%)(RNH₂+Na)⁺, 803 (15%)(RNH₃)⁺, 378 (45%), 132 (100%). M, 916.

### Example (23)

### 1-[3-(D-Alanyl-L-alanyl-L-alanyl-L-norleucyl-glycyl-L-leucyl-L-prolylamino)propylamino]anthraquinone trifluoroacetate. [Method E]

Prepared from the reaction of N-^{t}Boc-D-alanine-N-hydroxysuccinimide ester and 1-[3-(L-alanyl-L-alanyl-L-norleucyl-glycyl-L-leucyl-L-prolylamino)propylamino]-anthraquinone trifluoroacetate (22) in THF and triethylamine. Mp 192° C. FABMS(+) m/z: 897 (35%)(RNH₂+Na)⁺, 875 (65%)(RNH₃)⁺, 497 (12%), 378 (100%), 263 (52%). M, 988.

### Example (24)

### 1-[3-(S-Methyl-L-cnsteinyl-plycyl-L-leucyl-L-prolylamino)propylamino]anthraquinone trifluoroacetate. [Methods D and E]

N-^{t}Boc-S-methyl-L-cysteine was converted to its pentafluorophenolate ester and reacted with 1-[3-(glycyl-L-leucyl-L-prolylamino)propylamino]anthraquinone trifluoroacetate (4) in DMF and triethylamine. Mp 196° C. ESMS(+)(Cone 20V) m/z: 687 (70%)(RNH₂+Na)⁺, 665 (80%)(RNH₃)⁺, 413 (100%), 378 (25%), 279 (15%). ESMS(−) (Cone 20V) m/z: 113 (55%)(CF₃COO)⁻69 (100%). M, 778.

### Example (25)

### 1-[3-(L-Alanyl-S-methyl-L-cysteinyl-glycyl-L-leucyl-L-prolylamino)-propylamino]anthraguinone trifluoroacetate. [Method E]

Prepared from the reaction of N-^{t}Boc-L-alanine-N-hydroxysuccinimide ester and 1-[3-(S-methyl-L-cysteinyl-glycyl-L-leucyl-L-prolylamino)propylamino]anthraquinone trifluoroacetate (24) in THF and triethylamine. Mp 147° C. ESMS(+)(Cone 20V) m/z: 758 (15%)(RNH₂+Na)⁺, 736 (100%)(RNH₃)⁺, 413 (3%), 378 (5%). ESMS(−)(Cone 20V) m/z: 113 (100%)(CF₃COO)⁻69 (20%). M, 849.

### Example (26)

### 1-[3-(L-Alanyl-L-alanyl-S-methyl-L-cysteinyl-glycyl-L-leucyl-L-prolylamino)propylamino]anthraquinone trifluoroacetate. [Method E]

Prepared from the reaction of N-^{t}Boc-L-alanine-N-hydroxysuccinimide ester and 1-[3-(L-alanyl-S-methyl-L-cysteinyl-glycyl-L-leucyl-L-prolylamino)propylamino]-anthraquinone trifluoroacetate (25) in THF and triethylamine. Mp 162° C. ESMS(+)(Cone 20V) m/z: 829 (5%)(RNH₂+Na)⁺, 807 (100%)(RNH₃)⁺, 691 (5%), 562 (10%), 293 (15%). ESMS(−)(Cone 20V) m/z: 113 (100%)(CF₃COO)⁻69 (20%). M, 920.

### Example (27)

### 1-[3-(D-Alanyl-L-alanyl-L-alanyl-S-methyl-L-cysteinyl-glycyl-L-leucyl-L-prolylamino)propylamino]anthraquinone trifluoroacetate. [Methods D and E]

N-^{t}Boc-D-alanine was converted to its pentafluorophenolate ester and reacted with 1-[3-(L-alanyl-L-alanyl-S-methyl-L-cysteinyl-glycyl-L-prolylamino)propylamino]-anthraquinone trifluoroacetate (26) in DMF and triethylamine. Mp 191° C. ESMS(+)(Cone 90V) m/z: 900 (80%)(RNH₂+Na)⁺, 878 (40%)(RNH₃)⁺, 378 (100%), 102 (95%). ESMS(−)(Cone 20V) m/z: 113 (100%)(CF₃COO)⁻69 (30%). M, 991.

### Example (28)

### 1-[3-(L-Tyrosyl-L-leucyl-glycyl-L-leucyl-L-prolylamino)propylamino]-anthraquinone trifluoroacetate. [Method F]

Prepared by the reaction of N-Fmoc-O-tert-butyl-L-tyrosine pentafluorophenolate ester with 1-[3-(L-leucyl-glycyl-L-leucyl-L-prolylamino)propylamino]anthraquinone trifluoroacetate (5) in THF and triethylamine [note: after Fmoc deprotection the solid was dissolved in trifluoroacetic acid for 24 hours (to effect O-deprotection of the tyrosyl residue) before evaporation to give the title compound]. Mp 184-188° C. FABMS(+) m/z: 846 (4%)(RNH₂+Na)⁺, 824 (100%)(RNH₃)⁺. M, 937.

### Example (29)

### 1-[3-(L-Alanyl-L-tyrosyl-L-leucyl-glycyl-L-leucyl-L-prolylamino)-propylamino]anthraquinone trifluoroacetate. [Method F]

Prepared by the reaction of N-Fmoc-L-alanine pentafluorophenolate ester with 1-[3-(O-tert-butyl-L-tyrosyl-L-leucyl-glycyl-L-leucyl-L-prolylamino)propylamino]-anthraquinone trifluoroacetate in DMF and triethylamine [note: after Fmoc deprotection the solid was dissolved in trifluoroacetic acid for 24 hours (to effect O-deprotection of the tyrosyl residue) before evaporation to give the title compound]. Mp 182° C. ESMS(+)(Cone 20V) m/z: 917 (5%)(RNH₂+Na)⁺, 895 (100%)(RNH₃)⁺, 378 (2%). ESMS(−)(Cone 20V) m/z: 113 (60%)(CF₃COO)⁻69 (100%). M, 1008.

### Example (30)

### 1-[3-(D-Alanyl-L-alanyl-L-tyrosyl-L-leucyl-glycyl-L-leucyl-L-prolylamino)propylamino]anthraquinone trifluoroacetate. [Method F]

Prepared by the reaction of N-Fmoc-D-alanine pentafluorophenolate ester with 1-[3-(L-alanyl-O-tertiarybutyl-L-tyrosyl-L-leucyl-glycyl-L-leucyl-L-prolylamino)propylamino]-anthraquinone trifluoroacetate in DMF and triethylanine [note: after Fmoc deprotection the solid was dissolved in trifluoroacetic acid for 24 hours (to effect O-deprotection of the tyrosyl residue) before evaporation to give the title compound]. Mp 190-194° C. FABMS(+) m/z: 988 (10%)(RNH₂+Na)⁺, 966 (100%)(RNH₃)⁺, 378 (30%). M, 1079.

### Example (31)

### 1-[3-(L-Norvalyl-L-leucyl-glycyl-L-leucyl-L-prolylamino)propylamino]-anthraquinone trifluoroacetate. [Methods D and E]

N-^{t}Boc-L-norvaline was converted to its pentafluorophenolate ester and reacted with 1-[3-(L-leucyl-glycyl-L-leucyl-L-prolylamino)propylamino]anthraquinone trifluoroacetate (5) in DMF and triethylamine. Mp 189° C. ESMS(+)(Cone 20V) m/z: 782 (20%)(RNH₂+Na)⁺, 760 (40%)(RNH₃)⁺, 378 (5%), 134 (20%), 102 (100%). ESMS(−) (Cone 20V) m/z: 113 (100%)(CF₃COO)⁻69 (60%). M, 873.

### Example (32)

### 1-[3-(L-Alanyl-L-norvalyl-L-leucyl-glycyl-L-leucyl-L-prolylamino)-propylamino]anthraquinone trifluoroacetate. [Method E]

Prepared from the reaction of N-^{t}Boc-L-alanine-N-hydroxysuccinimide ester and 1-[3-(L-norvalyl-L-leucyl-glycyl-L-leucyl-L-prolylamino)propylamino]anthraquinone trifluoroacetate (31) in THF and triethylamine. Mp 192° C. FABMS(+) m/z: 853 (10%)(RNH₂+Na)⁺, 831 (15%)(RNH₃)⁺, 443 (10%), 378 (100%), 341 (20%). M, 944.

### Example (33)

### 1-[3-(D-Alanyl-L-alanyl-L-norvalyl-L-leucyl-glycyl-L-leucyl-L-prolylamino)propylamino]anthraquinone trifluoroacetate. [Method E]

Prepared from the reaction of N-^{t}Boc-D-alanine-N-hydroxysuccinimide ester and 1-[3-(L-alanyl-L-norvalyl-L-leucyl-glycyl-L-leucyl-L-prolylamino)propylamino]-anthraquinone trifluoroacetate (32) in THF and triethylamine. Mp 174-178° C. ESMS(+)(Cone 20V) m/z: 924 (5%)(RNH₂+Na)⁺, 902 (100%)(RNH₃)⁺, 378 (2%), 263 (5%). M,1015.

### Example (34)

### 1-[3-(D-Lysyl-L-alanyl-L-alanyl-L-leucyl-glycyl-L-leucyl-L-prolylamino)propylamino]anthraquinone bis trifluoroacetate. [Method F]

N-α-Fmoc-N-ε-^{t}Boc-D-lysine was converted to its pentafluorophenolate ester and reacted with 1-[3-(L-alanyl-L-alanyl-L-leucyl-glycyl-L-leucyl-L-prolylamino)propylamino]anthraquinone trifluoroacetate (7) in DMF and triethylamine (note: after Fmoc deprotection the solid was dissolved in trifluoroacetic acid for 20 minutes before evaporation to give the title compound). Mp 196° C. ESMS(+)(Cone 20V) m/z: 932 (100%), 1158.

### Example (35)

### 1-[3-(S-p-Methoxybenzyl-L-cysteinyl-glycyl-L-leucyl-L-prolylamino)-propylamino]anthraquinone trifluoroacetate. [Methods D and E]

N-^{t}Boc-S-p-methoxybenzyl-L-cysteine was converted to its pentafluorophenolate ester and reacted with 1-[3-(glycyl-L-leucyl-L-prolylamino)propylamino]anthraquinone trifluoroacetate (4) in DMF and triethylamine. Mp 182° C. ESMS(+)(Cone 20V) m/z: 793 (40%)(RNH₂+Na)⁺, 771 (100%)(RNH₃)⁺, 413 (50%), 281 (50%), 239 (20%).ESMS(−) (Cone 20V) m/z: 113 (100%)(CF₃COO)⁻69 (10%). M, 884.

### Example (36)

### 1-[3-(L-Alanyl-S-p-methoxybenzyl-L-cysteinyl-zlycyl-L-leucyl-L-prolylamino)propylamino]anthraguinone trifluoroacetate. [Method F]

Prepared by the reaction of N-Fmoc-L-alanine pentafluorophenolate ester with 1-[3-(S-p-methoxybenzyl-L-cysteinyl-glycyl-L-leucyl-L-prolylamino)propylamino]-anthraquinone trifluoroacetate (35) in DMF and triethylamine. Mp 187° C. ESMS(+)(Cone 20V) m/z: 864 (10%)(RNH₂+Na)⁺, 842 (70%)(RNH₃)⁺, 380 (10%), 317 (5%), 102 (100%). ESMS(−)(Cone 20V) m/z: 113 (100%)(CF₃COO)⁻69 (20%). M, 955.

### Example (37)

### 1-[3-(L-Alanyl-L-alanyl-S-p-methoxybenzyl-L-cysteinyl-glycyl-L-leucyl-L-prolylamino)propylamino]anthraquinone trifluoroacetate. [method F]

Prepared by the reaction of N-Fmoc-L-alanine pentafluorophenolate ester with 1-[3-(L-alanyl-S-p-methoxybenzyl-L-cysteinyl-glycyl-L-leucyl-prolylamino)propylamino]anthraquinone trifluoroacetate (36) in DMF and triethylamine. Mp 182° C. ESMS(+)(Cone 20V) m/z: 935 (50%)(RNH₂+Na)⁺, 913 (85%)(RNH₃)⁺, 129 (70%), 97 (100%). ESMS(−)(Cone 20V) m/z: 113 (100%)(CF₃COO)⁻69 (30%). M, 1026.

### Example (38)

### 1-[3D-Alanyl-L-alanyl-L-alanyl-S-p-methoxybenzyl-L-cysteinyl-glycyl-L-leucyl-L-prolylamino)propylamino]anthraquinone trifluoroacetate. [method F]

Prepared by the reaction of N-Fmoc-D-alanine pentafluorophenolate ester with 1-[3-(L-alanyl-L-alanyl-S-p-methoxybenzyl-L-cysteinyl-glycyl-L-leucyl-L-prolylamino)propylamino]anthraquinone trifluoroacetate (37) in DMF and triethylamine. Mp 190-192° C. ESMS(+)(Cone 20V) m/z: 1006 (5%)(RNH₂+Na)⁺, 984 (100%)(RNH₃)⁺. ESMS(−)(Cone 20V) m/z: 113 (70%)(CF₃COO)⁻69 (100%). M, 1097.

### Example (39)

### 1-[3-(L-Tyrosyl-L-norvalyl-glycyl-L-leucyl-L-prolylamino)propylamino]anthraquinone trifluoroacetate. [Methods F]

Prepared by the reaction of N-Fmoc-O-tert-butyl-L-tyrosine pentafluorophenolate ester with 1-[3-(L-norvalyl-glycyl-L-leucyl-L-prolyl-amino)propylamino]anthraquinone trifluoroacetate (16) in DMF and triethylamine [note: after Fmoc deprotection the solid was dissolved in trifluoroacetic acid for 24 hours (to effect O-deprotection of the tyrosyl residue) before evaporation to give the title compound]. Mp 174° C. ESMS(+)(Cone 20V) m/z: 832 (10%)(RNH₂+Na)⁺, 810 (100%)(RNH₃)⁺, 378 (5%). M, 923.

### Example (40)

### 1-[3-(L-Alanyl-L-tyrosyl-L-norvalyl-glycyl-L-leucyl-L-prolylamino)-propylamino]anthraquinone trifluoroacetate. [Methods F]

Prepared by the reaction of N-Fmoc-L-alanine pentafluorophenolate ester with 1-[3-(O-tertiarybutyl-L-tyrosyl-L-norvalyl-glycyl-L-leucyl-L-prolyl-amino)propylamino]-anthraquinone trifluoroacetate in DMF and triethylamine [note: after Fmoc deprotection the solid was dissolved in trifluoroacetic acid for 24 hours (to effect O-deprotection of the tyrosyl residue) before evaporation to give the title compound]. Mp 182° C. ESMS(+)(Cone 20V) m/z: 881 (100%)(RNH₃)⁺, 378 (2%). M, 994.

### Example (41)

### 1-[3-(D-Alanyl-L-alanyl-L-tyrosyl-L-norvalyl-glycyl-L-leucyl-L-prolylamino)propylamino]anthraquinone trifluoroacetate. [Method F]

Prepared by the reaction of N-Fmoc-D-alanine pentafluorophenolate ester with 1-[3-(L-alanyl-O-tertiarybutyl-L-tyrosyl-L-norvalyl-glycyl-L-leucyl-L-prolyl-amino)propylamino]anthraquinone trifluoroacetate in DMF and triethylamine [note: after Fmoc deprotection the solid was dissolved in trifluoroacetic acid for 24 hours (to effect O-deprotection of the tyrosyl residue) before evaporation to give the title compound]. Mp 190° C. ESMS(+)(Cone 20V) m/z: 974 (10%)(RNH₂+Na)⁺, 952 (100%)(RNH₃)⁺, 378 (5%). ESMS(−)(Cone 20V) m/z: 113 (100%)(CF₃COO)⁻69 (30%). M, 1065.

### Example (42)

### 1-[3-(L-Tyrosyl-S-methyl-L-cysteinyl-glycyl-L-leucyl-L-prolylamino)-propylamino]anthraquinone trifluoroacetate. [Method F]

Prepared by the reaction of N-Fmoc-O-tert-butyl-L-tyrosine pentafluorophenolate ester with 1-[3-(S-methyl-L-cysteinyl-glycyl-L-leucyl-L-prolylamino)propylamino]-anthraquinone trifluoroacetate (24) in DMF and triethylamine [note: after Fmoc deprotection the solid was dissolved in trifluoroacetic acid for 24 hours (to effect O-deprotection of the tyrosyl residue) before evaporation to give the title compound]. Mp 178-182° C. ESMS(+)(Cone 20V) m/z: 840 (3%)(RNH₂+Na)⁺, 828 (100%)(RNH₃)⁺. M, 941.

### Example (43)

### 1-[3-(L-Alanyl-L-tyrosyl-S-methyl-L-cysteinyl-glycyl-L-leucyl-L-prolyl-amino)propylamino]anthraquinone trifluoroacetate. [Method F]

Prepared by the reaction of N-Fmoc-L-alanine pentafluorophenolate ester with 1-[3-(O-tertiarybutyl-L-tyrosyl-S-methyl-L-cysteinyl-glycyl-L-leucyl-L-prolylamino)propyl-amino]anthraquinone trifluoroacetate) in DMF and triethylamine [note: after Fmoc deprotection the solid was dissolved in trifluoroacetic acid for 24 hours (to effect O-deprotection of the tyrosyl residue) before evaporation to give the title compound]. Mp 166-170° C. FABMS(+) m/z: 921 (15%)(RNH₂+Na)⁺, 899 (25%)(RNH₃)⁺, 378 (100%). M, 1012.

### Example (44)

### 1 -[3-(D-Alanyl-L-alanyl-L-tyrosyl-S-methyl-L-cysteinyl-glycyl-L-leucyl-L-prolylamino)propylamino]anthraquinone trifluoroacetate. [Method F]

Prepared by the reaction of N-Fmoc-D-alanine pentafluorophenolate ester with 1-[3-(L-alanyl-O-tertiarybutyl-L-tyrosyl-S-methyl-L-cysteinyl-glycyl-L-leucyl-L-prolyl-amino)-propylamino]anthraquinone trifluoroacetate) in DMF and triethylamine [note: after Fmoc deprotection the solid was dissolved in trifluoroacetic acid for 24 hours (to effect O-deprotection of the tyrosyl residue) before evaporation to give the title compound]. Mp 195° C. ESMS(+)(Cone 20V) m/z: 992 (5%)(RNH₂+Na)⁺, 970 (100%)(RNH₃)⁺. M, 1083.

### Example (45)

### 1-[3-(L-Norvalyl-L-norvalyl-glycyl-L-leucyl-L-prolylamino)propyl-amino]anthraquinone trifluoroacetate. [methods D and E]

N-^{t}Boc-L-norvaline was converted to its pentafluorophenolate ester and reacted with 1-[3-(L-norvalyl-glycyl-L-leucyl-L-prolylamino)propylamino]anthraquinone trifluoroacetate (16) in DMF and triethylamine. Mp 174-176° C. ESMS(+)(Cone 20V) m/z: 768 (15%)(RNH₂+Na)⁺, 746 (100%)(RNH₃)⁺, 378 (5%). M, 859.

### Example (46)

### 1-3-(L-Alanyl-L-norvalyl-L-norvalyl-glycyl-L-leucyl-L-prolylamino)-propylamino]anthraquinone trifluoroacetate. [Method E]

Prepared from the reaction of N-^{t}Boc-L-alanine-N-hydroxysuccinimide ester and 1-[3-(L-norvalyl-L-norvalyl-glycyl-L-leucyl-L-prolylamino)propyl-amino]anthraquinone trifluoroacetate (45) in THF and triethylamine. Mp 196° C. ESMS(+)(Cone 20V) m/z: 839 (10%)(RNH₂+Na)⁺, 817 (100%)(RNH₃)⁺, 378 (2%). ESMS(−)(Cone 20V) m/z: 113 (40%)(CF₃COO)⁻69 (100%). M, 930.

### Example (47)

### 1-[3-(D-AlanyI-L-alanyl-L-norvalyl-L-norvalyl-glycyl-L-leucyl-L-prolylamino)propylamino]anthraquinone trifluoroacetate. [Method E]

Prepared from the reaction of N-^{t}Boc-D-alanine-N-hydroxysuccinimide ester and 1-[3-(L-alanyl-L-norvalyl-L-norvalyl-glycyl-L-leucyl-L-prolylamino)propylamino]-anthraquinone trifluoroacetate (46) in THF and triethylamine. Mp 203° C. ESMS(+)(Cone 20V) m/z: 910 (20%)(RNH₂+Na)⁺, 888 (80%)(RNH₃)⁺, 413 (20%), 178 (30%), 114 (100%). ESMS(−)(Cone 20V) m/z: 113 (40%)(CF₃COO)⁻69 (100%). M, 1001.

### Example (48)

### 1-[3-(L-Norvalyl-L-prolylamino)propylamino]anthraquinone trifluoroacetate. [Methods D and E]

N-^{t}Boc-L-norvaline was converted to its pentafluorophenolate ester and reacted with 1-[3-(L-prolylamino)propylamino]anthraquinone trifluoroacetate (2) in DMF and triethylamine. Mp 190-194° C. ESMS(+)(Cone 20V) m/z: 499 (4%)(RNH₂+Na)⁺, 477 (100%)(RNH₃)⁺. ESMS(−)(Cone 20V) m/z: 113 (80%)(CF₃COO)⁻69 (100%). M, 590.

### Example (49)

### 1-[3-(Glycyl-L-norvalyl-L-prolylamino)propylamino]anthraquinone trifluoroacetate. [Method E]

Prepared by the reaction of N-^{t}Boc-glycine-N-hydroxysuccinimide ester with 1-[3-(L-norvalyl-L-prolylamino)propylamino]anthraquinone trifluoroacetate (48) in THF and triethylamine. Mp 196° C. ESMS(+)(Cone 50V) m/z: 556 (3%)(RNH₂+Na)⁺, 534 (50%)(RNH₃)⁺, 378 (100%). ESMS(−)(Cone 50V) m/z: 113 (20%)(CF₃COO)⁻ 69 (100%). M, 647.

### Example (50)

### 1-[3-(L-Leucyl-glycyl-L-norvalyl-L-prolylamino)propylamino]-anthraquinone trifluoroacetate. [Methods D and E]

N-^{t}Boc-L-leucylglycine was converted to its pentafluorophenolate ester and reacted with 1-[3-(glycyl-L-norvalyl-L-prolylamino)propylamino]anthraquinone trifluoroacetate (49) in DMF and triethylamine. Mp 172-176° C. ESMS(+)(Cone 20V) m/z: 669 (10%)(RNH₂+Na)⁺, 647 (100%)(RNH₃)⁺. ESMS(−)(Cone 20V) m/z: 113 (30%)(CF₃COO)⁻69 (100%). M, 760.

### Example (51)

### 1-[3-(L-Alanyl-L-leucyl-glycyl-L-norvalyl-L-prolylamino)propylamino]-anthraquinone trifluoroacetate. [Method E]

Prepared from the reaction of N-^{t}Boc-L-alanine-N-hydroxysuccinimide ester and 1-[3-(L-leucyl-glycyl-L-norvalyl-L-prolylamino)propylamino]anthraquinone trifluoroacetate (50) in THF and triethylamine. Mp 176-178° C. ESMS(+)(Cone 20V) m/z: 740 (3%)(RNH₂+Na)⁺, 718 (100%)(RNH₃)⁺, 378 (2%). M, 831.

### Example (52)

### 1-[3-(L-Alanyl-L-alanyl-L-leucyl-glycyl-L-norvalyl-L-prolylamino)-propylamino]anthraquinone trifluoroacetate. [Method E]

Prepared from the reaction of N-^{t}Boc-L-alanine-N-hydroxysuccinimide ester and 1-[3-(L-alanyl-L-leucyl-glycyl-L-norvalyl-L-prolylamino)propylamino]anthraquinone trifluoroacetate. (51) in THF and triethylamine. Mp 188° C. ESMS(+)(Cone 20V) m/z: 811 (10%)(RNH₂+Na)⁺, 789 (100%)(RNH₃)⁺. M, 902.

### Example (53)

### 1-[3-(D-Alanyl-L-alanyl-L-alanyl-L-leucyl-glycyl-L-norvalyl-L-prolylamino)-propylamino]anthraquinone trifluoroacetate. [Method E]

Prepared from the reaction of N-^{t}Boc-D-alanine-N-hydroxysuccinimide ester and 1-[3-(L-alanyl-L-analyl-L-leucyl-glycyl-L-norvalyl-L-prolylamino)propylamino]-anthraquinone trifluoroacetate (52) in THF and triethylamine. Mp 192-196° C. ESMS(+)(Cone 20V) m/z: 882 (3%/)(RNH₂+Na)⁺, 860 (100%)(RNH₃)⁺. ESMS(−)(Cone 20V) m/z: 113 (100%)(CF₃COO)⁻69 (65%). M, 973.

### Example (54)

### 1-[3-(L-Alanyl-L-leucyl-L-prolylamino)propylamino]anthraquinone trifluoroacetate. [Method E]

Prepared from the reaction of N-^{t}Boc-L-alanine-N-hydroxysuccinimide ester and 1-[3-(L-leucyl-L-prolylamino)propylamino]anthraquinone trifluoroacetate (3) in THF and triethylamine. Mp 170-174° C. FABMS(+) m/z: 584 (20%)(RNH₂+Na)⁺, 562 (30%)(RNR₃)⁺, 378 (100%). M, 675.

### Example (55)

### 1-[3-(L-Leucyl-L-alanyl-L-leucyl-L-prolylamino)propylamino]-anthraquinone trifluoroacetate. [Method E]

Prepared from the reaction of N-^{t}Boc-leucine-N-hydroxysuccinimide ester and 1-[3-(L-alanyl-L-leucyl-L-prolylamino)propylamino]anthraquinone trifluoroacetate (54) in THF and triethylamine. Mp 178-180° C. ESMS(+)(Cone 20V) m/z: 697 (5%)(RNH₂+Na)⁺, 675 (100%)(RNH₃)⁺. M, 788.

### Example (56)

### 1-[3-(Glycyl-L-leucyl-L-alanyl-L-leucyl-L-prolylamino)propylamino]-anthraquinone trifluoroacetate. [Method E]

Prepared from the reaction of N-^{t}Boc-glycine-N-hydroxysuccinimide ester and 1-[3-(L-leucyl-L-alanyl-L-leucyl-L-prolylamino)propylamino]anthraquinone trifluoroacetate (55) in THF and triethylamine. Mp 192-194° C. ESMS(+)(Cone 20V) m/z: 754 (15%)(RNH₂+Na)⁺, 732 (100%)(RNH₃)⁺. M, 845.

### Example (57)

### 1-[3-(L-Alanyl-glycyl-L-leucyl-L-alanyl-L-leucyl-L-prolylamino)propyl-amino]anthraquinone trifluoroacetate. [Method E]

Prepared from the reaction of N-^{t}Boc-L-alanine-N-hydroxysuccinimide ester and 1-[3-(glycyl-L-leucyl-L-alanyl-L-leucyl-L-prolylamino)propylamino]anthraquinone trifluoroacetate (56) in THF and triethylamine. Mp 185° C. ESMS(+)(Cone 20V) m/z: 825 (5%)(RNH₂+Na)⁺, 803 (100%)(RNH₃)⁺. ESMS(−)(Cone 20V) m/z: 113 (50%)(CF₃COO)⁻ 69 (100%). M, 916.

### Example (58)

### 1-[3-(D-Alanyl-L-alanyl-glycyl-L-leucyl-L-alanyl-L-leucyl-L-prolylamino)propylamino]anthraquinone trifluoroacetate. [Method E]

Prepared from the reaction of N-^{t}Boc-D-alanine-N-hydroxysuccinimide ester and 1-[3-(L-alanyl-glycyl-L-leucyl-L-alanyl-L-leucyl-L-prolylamino)propylamino]anthraquinone trifluoroacetate (57) in THF and triethylamine. Mp 192° C. ESMS(+)(Cone 20V) m/z: 896 (15%)(RNH₂+Na)⁺, 874 (100%)(RNH₃)⁺, 378 (3%). ESMS(−)(Cone 20V) m/z: 113 (70%)(CF₃COO)⁻ 69 (100%). M, 987.

### Example (59)

### 1-[3-(L-Phenylalanyl-L-leucyl-L-prolylamino)propylamino]-anthraquinone trifluoroacetate. [Method E]

Prepared from the reaction of N-^{t}Boc-L-phenylalanine-N-hydroxysuccinimide ester and 1-[3(L-leucyl-L-prolylamino)propylamino]anthraquinone trifluoroacetate (3) in THF and triethylamine. Mp 184-188° C. ESMS(+)(Cone 20V) m/z: 660 (10%)(RNH₂+Na)⁺, 638 (100%)(RNH₃)⁺. ESMS(−)(Cone 50V) m/z: 113 (30%)(CF₃COO)⁻ 69 (100%). M, 751.

### Example (60)

### 1-[3-(L-Leucyl-L-phenylalanyl-L-leucyl-L-prolylamino)propylamino]-anthraquinone trifluoroacetate. [Method E]

Prepared from the reaction of N-^{t}Boc-L-leucine-N-hydroxysuccinimide ester and 1-[3-(L-phenylalanyl-L-leucyl-L-prolylamino)propylamino]anthraquinone trifluoroacetate (59) in THF and triethylamine. Mp 184-186° C. ESMS(+)(Cone 20V) m/z: 773 (15%)(RNH₂+Na)⁺, 751 (100%)(RNH₃)⁺. ESMS(−)(Cone 20V) m/z: 113 (70%)(CF₃COO)⁻ 69 (100%). M, 864.

### Example (61)

### 1-[3-(Glycyl-L-leucyl-L-phenylalanyl-L-leucyl-L-prolylamino)propyl-amino]anthraquinone trifluoroacetate. [Method E]

Prepared from the reaction of N-^{t}Boc-glycine-N-hydroxysuccinimide ester and 1-[3-(L-leucyl-L-phenylalanyl-L-leucyl-prolylamino)propylamino]anthraquinone trifluoroacetate (60) in THF and triethylamine. Mp 192° C. ESMS(+)(Cone 90V) n/z: 830 (20%)(RNH₂+Na)⁺, 808 (60%)(RNH₃)⁺, 378 (100%). ESMS(−)(Cone 50V) m/z: 113 (40%)(CF₃COO)⁻ 69 (100%). M, 921.

### Example (62)

### 1 -[3-(L-Alanyl-glycyl-L-leucyl-L-phenylalanyl-L-leucyl-L-prolylamino)-propylamino]anthraquinone trifluoroacetate. [Method E]

Prepared from the reaction of N-^{t}Boc-L-alanine-N-hydroxysuccinimide ester and 1-[3-(glycyl-L-leucyl-L-phenylalanyl-L-leucyl-L-prolylamino)propylamino]anthraquinone trifluoroacetate (61) in TBF and triethylamine. Mp 186° C. ESMS(+)(Cone 20V) m/z: 901 (20%)(RNH₂+Na)⁺, 879 (60%)(RNH₃)⁺, 238 (5%). ESMS(−)(Cone 50V) m/z: 113 (75%)(CF₃COO)⁻ 69 (100%). M, 992.

### Example (63)

### 1-[3-(D-Alanyl-L-alanyl-glycyl-L-leucyl-L-phenylalanyl-L-leucyl-L-prolylamino)propylamino]anthraquinone trifluoroacetate. [Methods D and E]

N-^{t}Boc-D-alanine was converted to its pentafluorophenolate ester and reacted with 1-[3-(L-alanyl-glycyl-L-leucyl-L-phenylalanyl-L-leucyl-L-prolylamino)propylamino]-anthraquinone trifluoroacetate (62) in DMF and triethylamine. Mp 190-194° C. ESMS(+)(Cone 50V) m/z: 972 (20%)(RNH₂+Na)⁺, 950 (60%)(RNH₃)⁺, 378 (100%). ESMS(−)(Cone 50V) m/z: 113 (70%)(CF₃COO)⁻ 69 (100%). M, 1063.

### Example (64)

### 1-[3-(Glycyl-L-prolylamino)propylamino]anthraquinone trifluoroacetate. 5 [Method E]

Prepared from the reaction of N-^{t}Boc-L-glycine-N-hydroxysuccinimide ester and 1-[3-(L-prolylamino)propylamino]anthraquinone trifluoroacetate (2) in THF and triethylamine. Mp 160-166° C. ESMS(+)(Cone 20V) m/z: 457 (5%)(RNH₂+Na)⁺, 435 (100%)(RNH₃)⁺. ESMS(−)(Cone 50V) m/z: 113 (40%)(CF₃COO)⁻, 69 (100%). M, 548.

### Example (65)

### 1-[3-(L-Leucyl-glycyl-L-prolylamino)propylamino]anthraquinone trifluoroacetate. [Method E]

Prepared from the reaction of N-^{t}Boc-L-leucine-N-hydroxysuccinimide ester and 1-[3-(glycyl-L-prolylamino)propylamino]anthraquinone trifluoroacetate (64) in THF and triethylamine. Mp 172-174° C. ESMS(+)(Cone 20V) m/z: 548 (100%)(RNH₃)⁺. ESMS(−)(Cone 20V) m/z: 113 (100%)(CF₃COO)⁻. M, 661.

### Example (66)

### 1-[3-(L-Phenylalanyl-L-leucyl-glycyl-L-prolylamino)propylamino]-anthraquinone trifluoroacetate. [Method E]

Prepared from the reaction of N-^{t}Boc-L-phenylalanine-N-hydroxysuccinimide ester and 1-[3-(L-leucyl-glycyl-L-prolylamino)propylamino]anthraquinone trifluoroacetate (65) in THF and triethylamine. Mp 164-168° C. ESMS(+)(Cone 20V) m/z: 695 (100%)(RNH₃)⁺. ESMS(−)(Cone 20V) m/z: 113 (100%)(CF₃COO)⁻. M, 808.

### Example (67)

### 1 -[3-(Glycyl-L-phenylalanyl-L-leucyl-glycyl-L-prolylamino)propyl-amino]anthraquinone trifluoroacetate. [Method E]

Prepared from the reaction of N-^{t}Boc-L-glycine-N-hydroxysuccinimide ester and 1-[3-(L-phenylalanyl-L-leucyl-glycyl-L-prolylamino)propylamino]anthraquinone trifluoroacetate (66) in THF and triethylamine. Mp 157° C. ESMS(+)(Cone 20V) m/z: 752 (100%)(RNH₃)⁺. ESMS(−)(Cone 20V) m/z: 113 (90%)(CF₃COO)⁻, 69 (100%). M, 865.

### Example (68)

### 1-[3-(Glycyl-D-prolylamino)propylamino]anthraquinone trifluoroacetate. [Method E]

Prepared from the reaction of N-^{t}Boc-L-glycine-N-hydroxysuccinimide ester and 1-[3-(D-prolylamino)propylamino]anthraquinone trifluoroacetate (9) in THF and triethylamine. Mp 156-162° C. ESMS(+)(Cone 20V) m/z: 457 (10)(RNH₂+Na)⁺, 435 (100%)(RNH₃)⁺. ESMS(−)(Cone 20V) m/z: 113 (80%)(CF₃COO)⁻, 69 (100%). M, 548.

### Example (69)

### 1-[3-(L-Leucyl-glycyl-D-prolylamino)propylamino]anthraquinone trifluoroacetate. [Method E]

Prepared from the reaction of N-^{t}Boc-L-leucine-N-hydroxysuccinimide ester and 1-[3-(glycyl-D-prolylamino)propylamino]anthraquinone trifluoroacetate (68) in THF and triethylamine. .Mp 174-178° C. ESMS(+)(Cone 20V) m/z: 548 (100%)(RNH₃)⁺. ESMS(−)(Cone 20V) m/z: 113 (100%)(CF₃COO)⁻. M, 661.

### Example (70)

### 1-[3-(L-Phenylalanyl-L-leucyl-glycyl-D-prolylamino)propylamino]-anthraquinone trifluoroacetate. [Method E]

Prepared from the reaction of N-^{t}Boc-L-phenylalanine-N-hydroxysuccinimide ester and 1-[3-(L-leucyl-glycyl-D-prolylamino)propylamino]anthraquinone trifluoroacetate (69) in THF and triethylamine. Mp 170° C. ESMS(+)(Cone 20V) m/z: 695 (100%)(RNH₃)⁺. ESMS(−)(Cone 20V) m/z: 113 (100%)(CF₃COO)⁻. M, 808.

### Example (71)

### 1-[3-(Glycyl-L-phenylalanyl-L-leucyl-glycyl-D-prolylamino)propyl-amino]anthraquinone trifluoroacetate. [Method E]

Prepared from the reaction of N-^{t}Boc-L-glycine-N-hydroxysuccinimide ester and 1-[3-(L-phenylalanyl-L-leucyl-glycyl-D-prolylamino)propylamino]anthraquinone trifluoroacetate (70) in THF and triethylanine. Mp 154-156° C. ESMS(+)(Cone 20V) m/z: 752 (100%)(RNH₃)⁺. ESMS(−)(Cone 20V) m/z: 113 (90%)(CF₃COO)⁻, 69 (100%). M, 865.

### Example (72)

### 1-[3-(L-Phenylalanyl-L-prolylamino)propylamino]anthraquinone trifluoroacetate. [Method E]

Prepared from the reaction of N-^{t}Boc-L-phenylalanine-N-hydroxysuccinimide ester and 1-[3-(L-prolylamino)propylamino]anthraquinone trifluoroacetate (2) in THF and triethylamine. Mp 118-120° C. ESMS(+)(Cone 20V) m/z: 547 (10)(RNH₂+Na)⁺, 525 (100%)(RNH₃)⁺. ESMS(−)(Cone 20V) m/z: 113 (100%)(CF₃COO)⁻. M, 638.

### Example (73)

### 1-[3-(Glycyl-L-phenylalanyl-L-prolylamino)propylamino]anthraquinone trifluoroacetate. [Methods E]

Prepared from the reaction of N-^{t}Boc-glycine-N-hydroxysuccinimide ester and 1-[3-(L-phenylalanyl-L-prolylamino)propylamino]anthraquinone trifluoroacetate (72) in THF and triethylamine. Mp 129° C. ESMS(+)(Cone 20V) m/z: 582 (100%)(RNH₃)⁺. ESMS(−)(Cone 20V) m/z: 113 (100%)(CF₃COO)⁻. M, 695.

### Example (74)

### 1-[3-(L-Isoleucyl-glycyl-L-phenylalanyl-L-prolylamino)propylamino]-anthraquinone trifluoroacetate. [Method E]

Prepared from the reaction of N-^{t}Boc-isoleucine-N-hydroxysuccinimide ester and 1-[3-(glycyl-L-phenylalanyl-L-prolylamino)propylamino]anthraquinone trifluoroacetate (73) in THF and triethylamine. Mp 122-124° C. ESMS(+)(Cone 20V) m/z: 695 (100%)(RNH₃)⁺. ESMS(−)(Cone 20V) m/z: 113 (100%)(CF₃COO)⁻. M, 808.

### Example (75)

### 1-[3-(L-Phenylalanyl-L-isoleucyl-glycyl-L-phenylalanyl-L-prolylamino)-propylamino]anthraquinone trifluoroacetate. [Method E]

Prepared from the reaction of N-^{t}Boc-L-phenylalanine-N-hydroxysuccinimide ester and 1-[3-(L-isoleucyl-glycyl-L-phenylalanyl-L-prolylamino)propylamino]anthraquinone trifluoroacetate (74) in THF and triethylamine. Mp 132-136° C. ESMS(+)(Cone 20V) m/z: 842 (100%)(RNH₃)⁺. ESMS(−)(Cone 20V) m/z: 113 (100%)(CF₃COO)⁻. M, 955.

### Example (76)

### 1-[3-(L-Leucyl-L-phenylalanyl-L-isoleucyl-glycyl-L-phenylalanyl-L-prolylamino)propylamino]anthraquinone trifluoroacetate. [Methods E]

Prepared from the reaction of N-^{t}Boc-L-leucine-N-hydroxysuccinimide ester and 1-[3-(L-phenylalanyl-L-isoleucyl-glycyl-L-phenylalanyl-L-prolylamino)propylamino]-anthraquinone trifluoroacetate (75) in THF and triethylamine. Mp 166-178° C. ESMS(+)(Cone 20V) m/z: 955 (100%)(RNH₃)⁺. ESMS(−)(Cone 20V) m/z: 113 (100%)(CF₃COO)⁻. M, 1068.

### Example (77)

### 1-[3-(D-Alanyl-L-Leucyl-L-Phenylalanyl-L-isoleucyl-glycyl-L-phenylalanyl-L-prolylamino)propylamino]anthraquinone trifluoroacetate. [Methods D and E]

N-^{t}Boc-D-alanine was converted to its pentafluorophenolate ester and reacted with 1-[3-(L-leucyl-L-phenylalanyl-L-isoleucyl-glycyl-L-phenylalanyl-L-prolylamino)propyl-amino]anthraquinone trifluoroacetate (76) in DMF and triethylamine. Mp 142-148° C. ESMS(+)(Cone 20V) m/z: 1026 (100%)(RNH₃)⁺. ESMS(−)(Cone 20V) m/z: 113 (100%)(CF₃COO)⁻. M, 1139.

### Example (78)

### 1-[3-(L-Phenylalanyl-D-prolylamino)propylamino]anthraquinone trifluoroacetate. [Methods D and E]

N-^{t}Boc-L-phenylalanine was converted to its pentafluorophenolate ester and reacted with 1-[3-(D-prolylamino)propylamino]anthraquinone trifluoroacetate (9) in THF and triethylamine. Mp 118-120° C. ESMS(+)(Cone 20V) m/z: 525 (100%)(RNH₃)⁺. ESMS(−)(Cone 20V) m/z: 113 (100%)(CF₃COO)⁻. M, 638.

### Example (79)

### 1-[3-(Glycyl-L-phenylalanyl-D-prolylamino)propylamino]anthraquinone trifluoroacetate. [Method E]

Prepared from the reaction of N-^{t}Boc-glycine-N-hydroxysuccinimide ester and 1-[3-(L-phenylalanyl-D-prolylamino)propylamino]anthraquinone trifluoroacetate (78) in THF and triethylamine. Mp 128-130° C. ESMS(+)(Cone 20V) m/z: 582 (100%)(RNH₃)⁺. ESMS(−)(Cone 20V) m/z: 113 (100%)(CF₃COO)⁻. M, 695.

### Example (80)

### 1-[3-(L-Isoleucyl-glycyl-L-phenylalanyl-D-prolylamino)propylamino]-anthraquinone trifluoroacetate. [Methods D and E]

N-^{t}Boc-isoleucine was converted to its pentafluorophenolate ester and reacted with 1-[3-(glycyl-L-phenylalanyl-D-prolylamino)propylamino]anthraquinone trifluoroacetate (79) in DMF and triethylamine. Mp 120-126° C. ESMS(+)(Cone 20V) m/z: 695 (100%)(RNH₃)⁺. ESMS(−)(Cone 20V) m/z: 113 (100%)(CF₃COO)⁻. M, 808.

### Example (81)

### 1-[3-(L-Phenylalanyl-L-isoleucyl-glycyl-L-phenylalanyl-D-prolylamino)-propylamino]anthraquinone trifluoroacetate. [Method E]

Prepared from the reaction of N-^{t}Boc-L-phenylalanine-N-hydroxysuccinimide ester and 1-[3-(L-isoleucyl-glycyl-L-phenylalanyl-D-prolylamino)propylamino]anthraquinone trifluoroacetate (80) in THF and triethylamine. Mp 130-134° C. ESMS(+)(Cone 20V) m/z: 842 (100)(RNH₃)⁺. ESMS(−)(Cone 20V) m/z: 113 (100%)(CF₃COO)⁻. M, 955.

### Example (82)

### 1-[3-(L-Leucyl-L-phenylalanyl-L-isoleucyl-glycyl-L-phenylalanyl-D-prolylamino)propylamino]anthraquinone trifluoroacetate. [Method E]

Prepared from the reaction of N-^{t}Boc-L-leucine-N-hydroxysuccinimide ester and 1-[3-(L-phenylalanyl-L-isoleucyl-glycyl-L-phenylalanyl-D-prolylamino)propylamino]-anthraquinone trifluoroacetate (81) in THF and triethylamine. Mp 168-170° C. ESMS(+)(Cone 20V) m/z: 955 (100%)(RNH₃)⁺. ESMS(−)(Cone 20V) m/z: 113 (100%)(CF₃COO)⁻. M, 1068.

### Example (83)

### 1-[3-(D-Alanyl-L-leucyl-L-phenylalanyl-L-isoleucyl-glycyl-L-phenylalanyl-D-prolylamino)propylamino]anthraquinone trifluoroacetate. [Methods D and E]

N-^{t}Boc-D-alanine was converted to its pentafluorophenolate ester and reacted with 1-[3-(L-leucyl-L-phenylalanyl-L-isoleucyl-glycyl-L-phenylalanyl-D-prolylamino)propyl-amino]anthraquinone trifluoroacetate (82) in DMF and triethylamine. Mp 144-146° C. ESMS(+)(Cone 20V) m/z: 1026 (100%)(RNH₃)⁺. ESMS(−)(Cone 20V) m/z: 113 (100%)(CF₃COO)⁻. M, 1139.

Biological Activity Studies

### On Example 8 [NU:UB187] and Metabolites: Topoisomerase Interaction and DNA Binding Properties

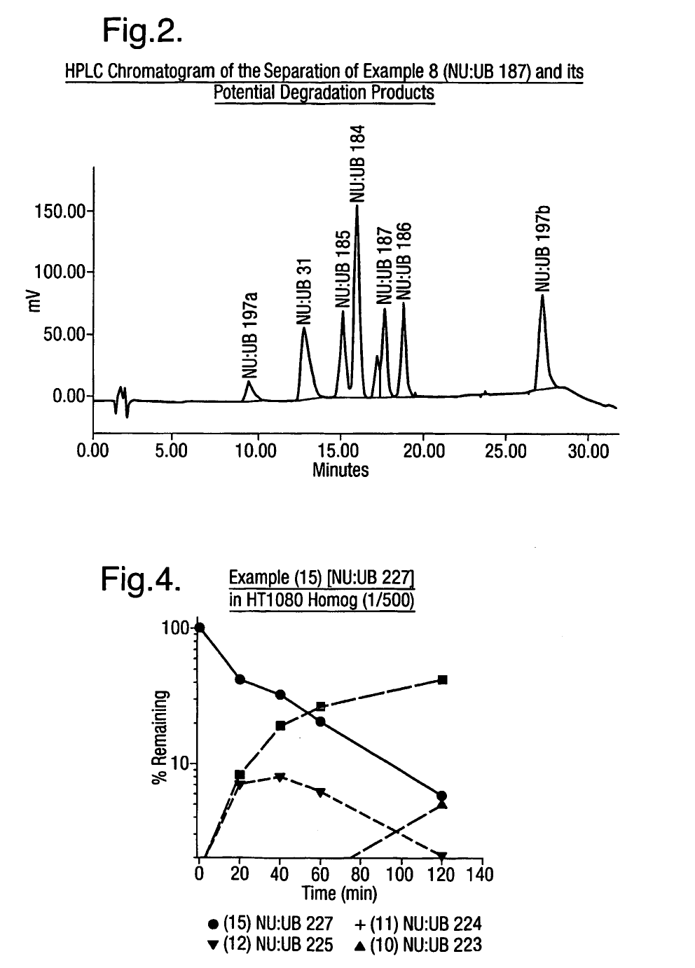

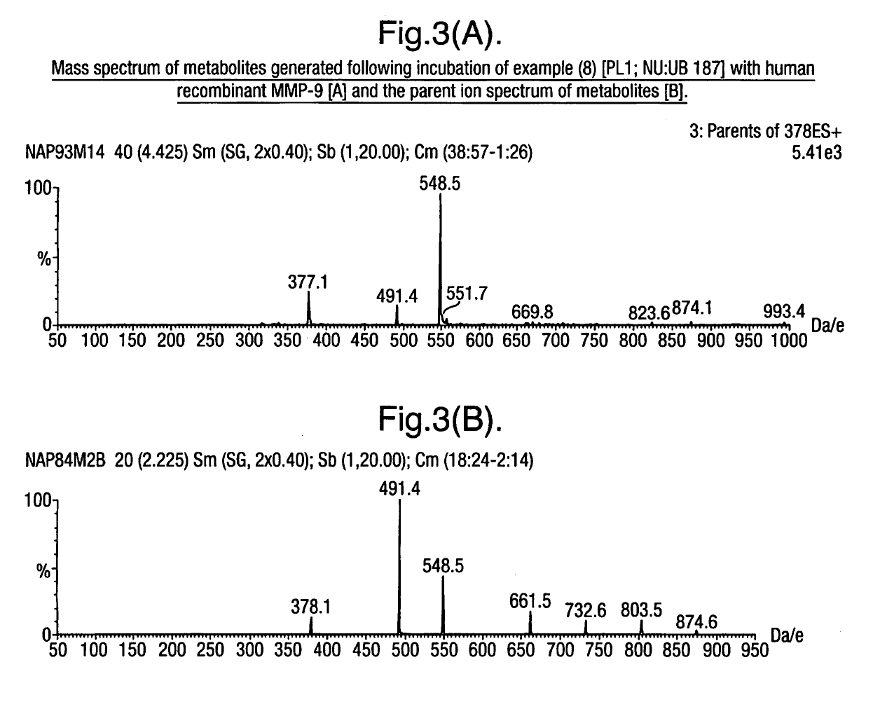

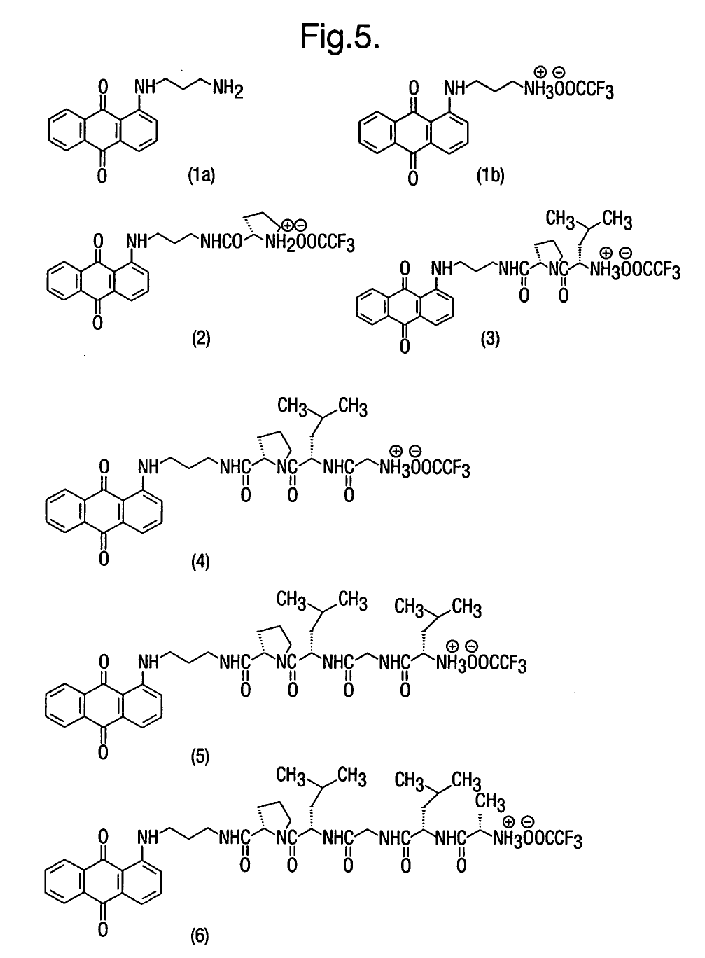

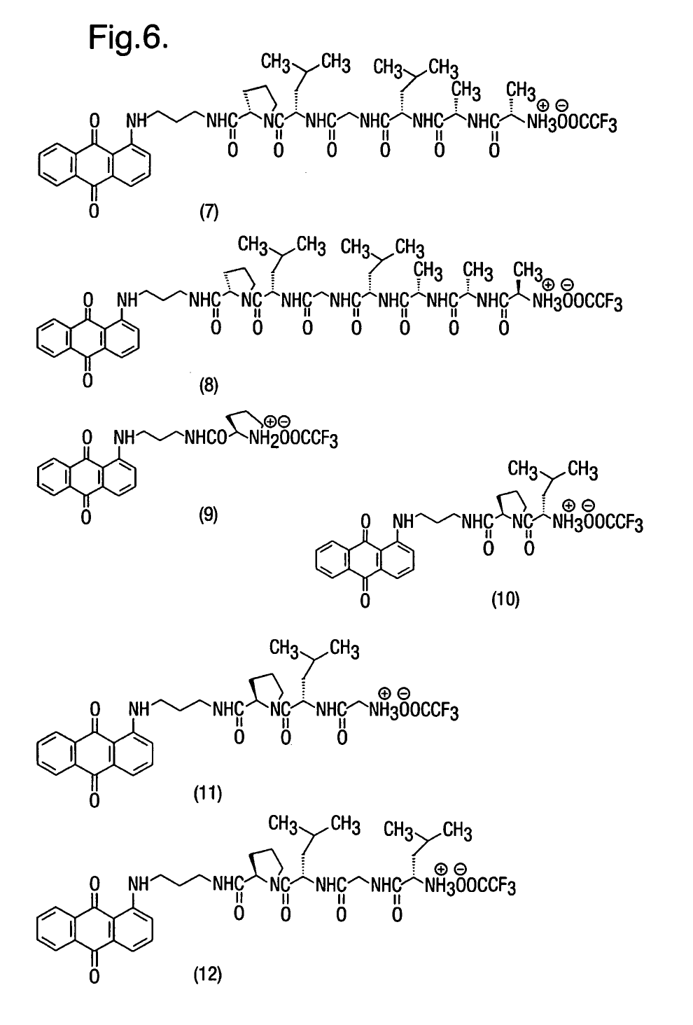

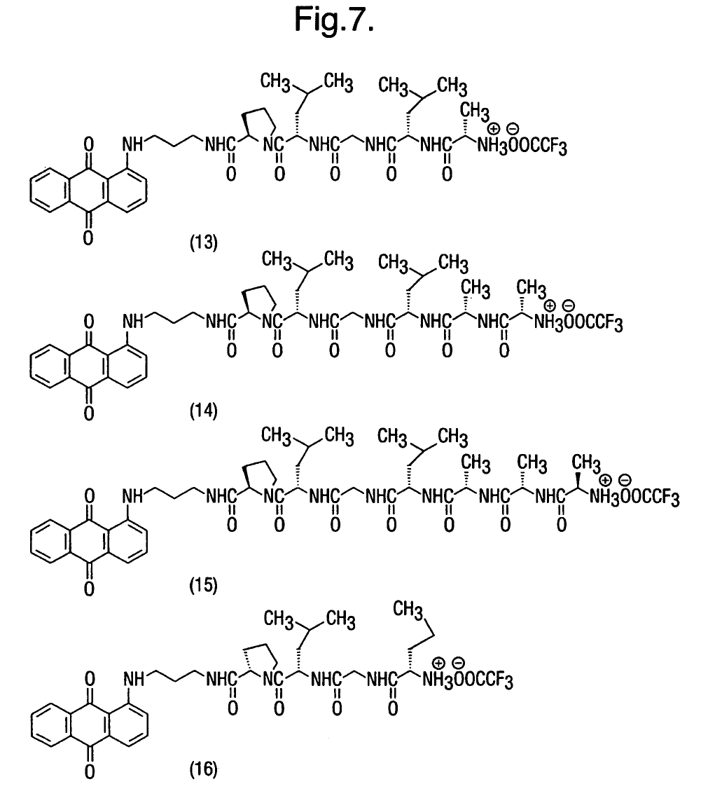

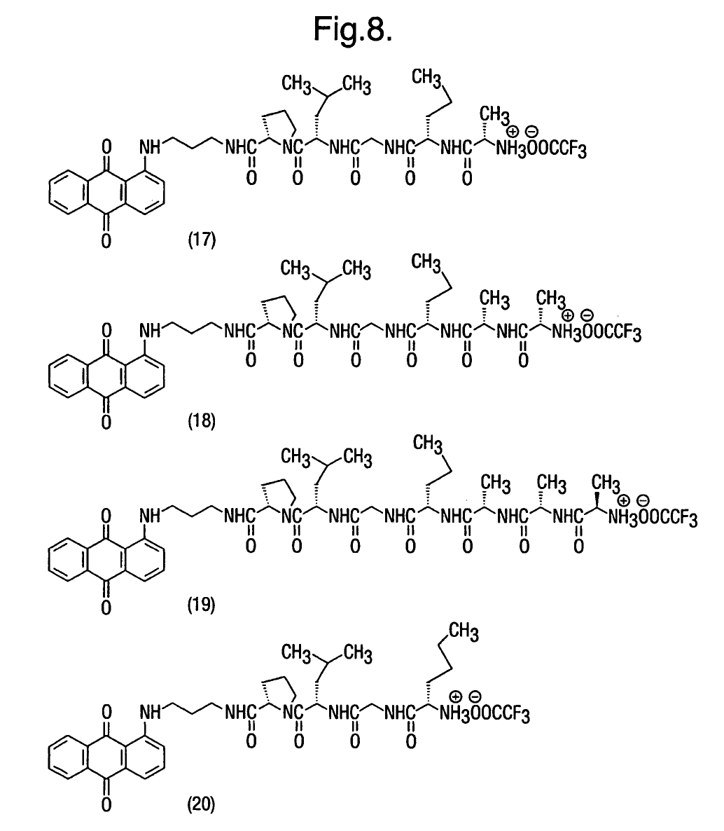

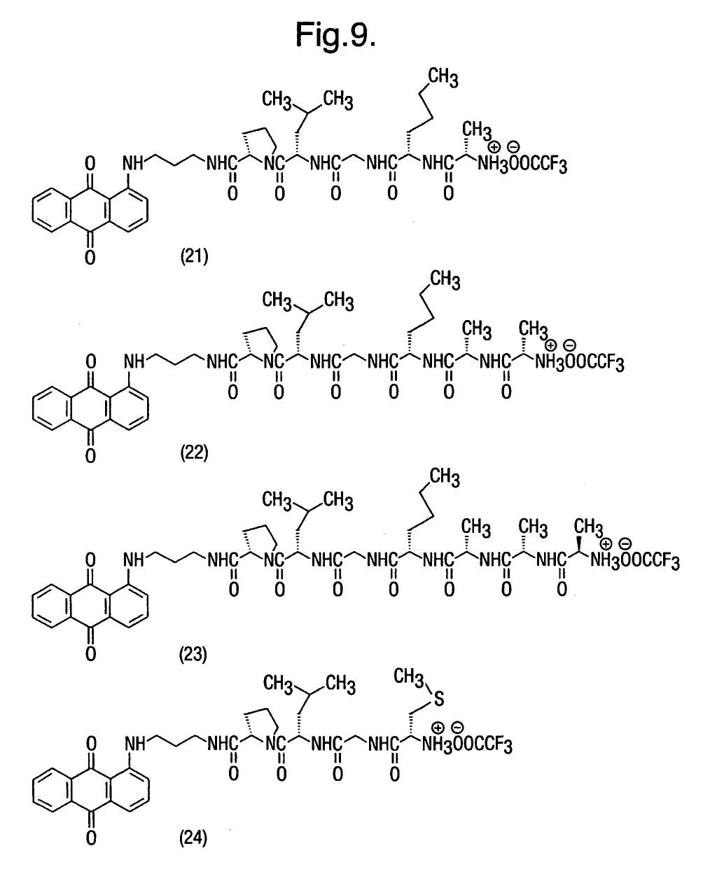

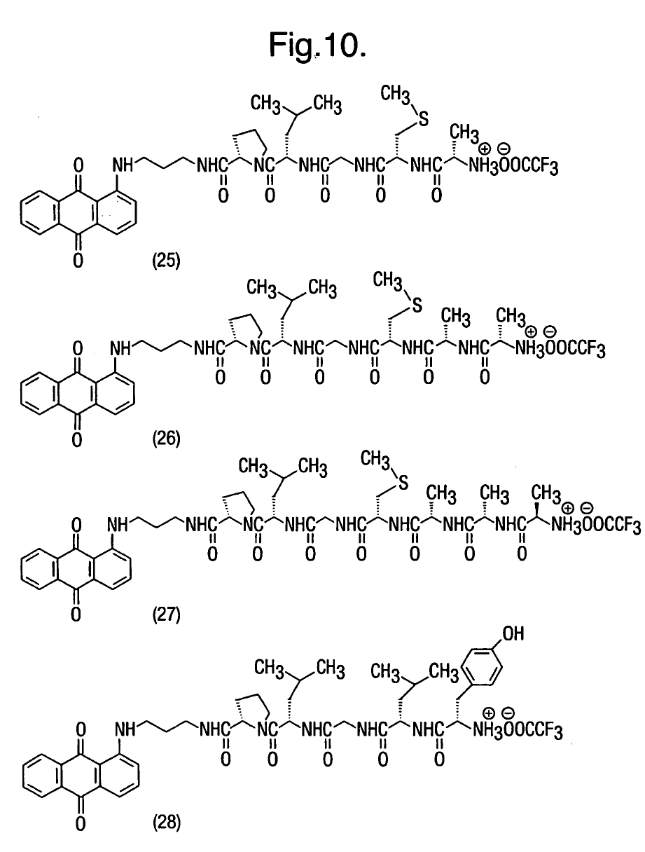

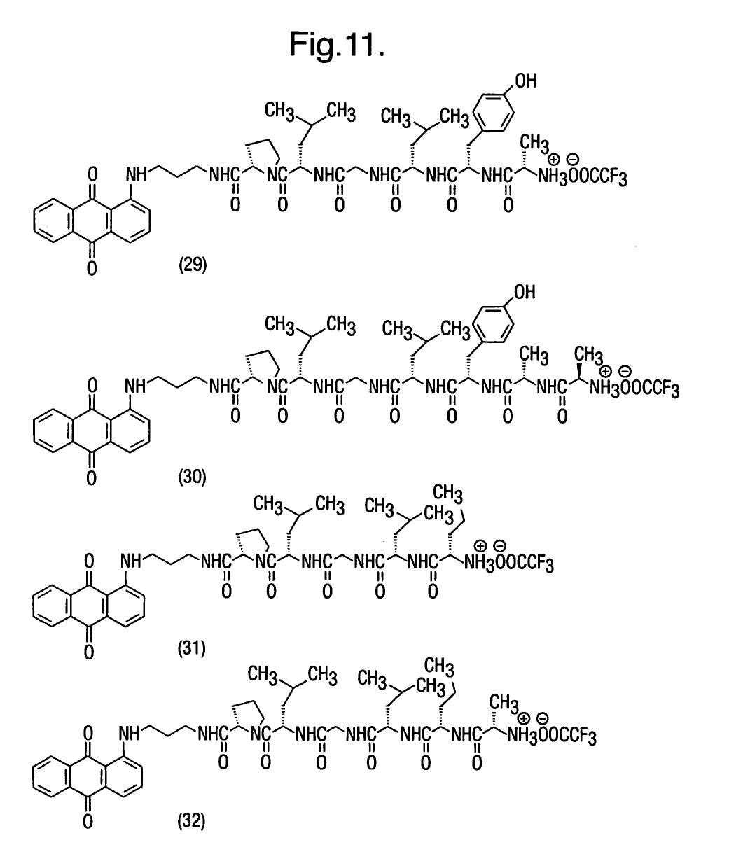

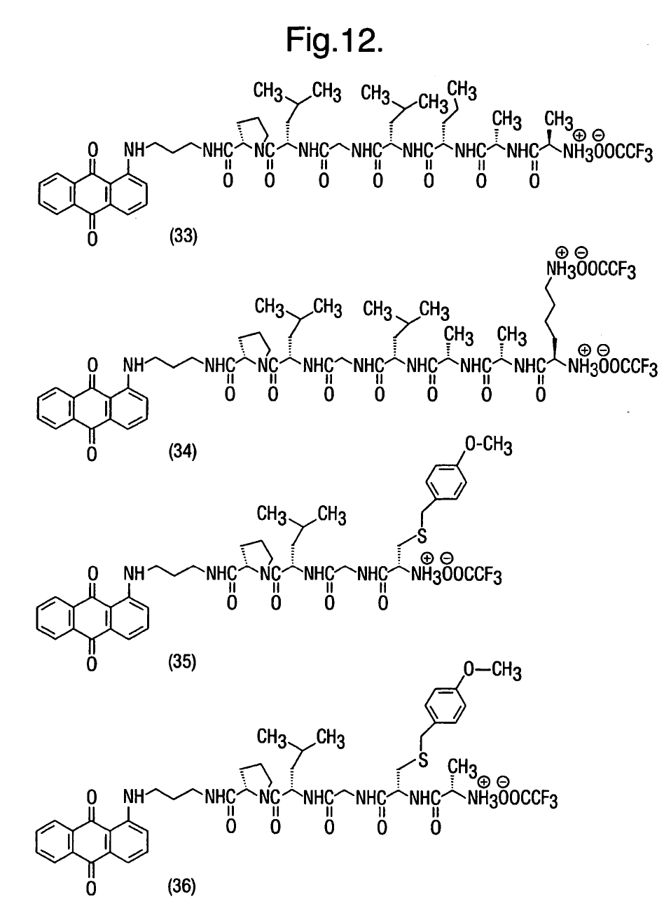

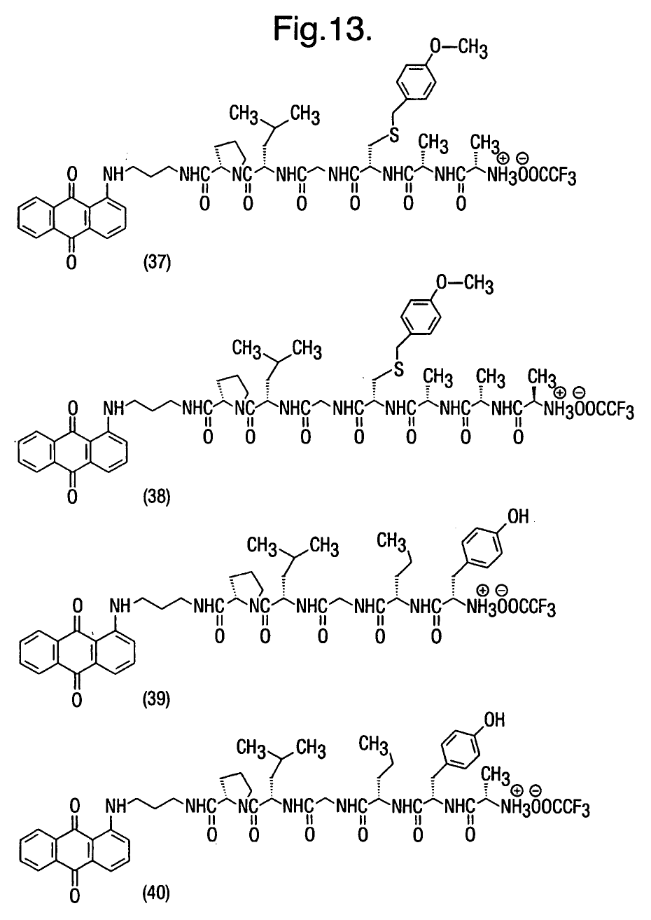

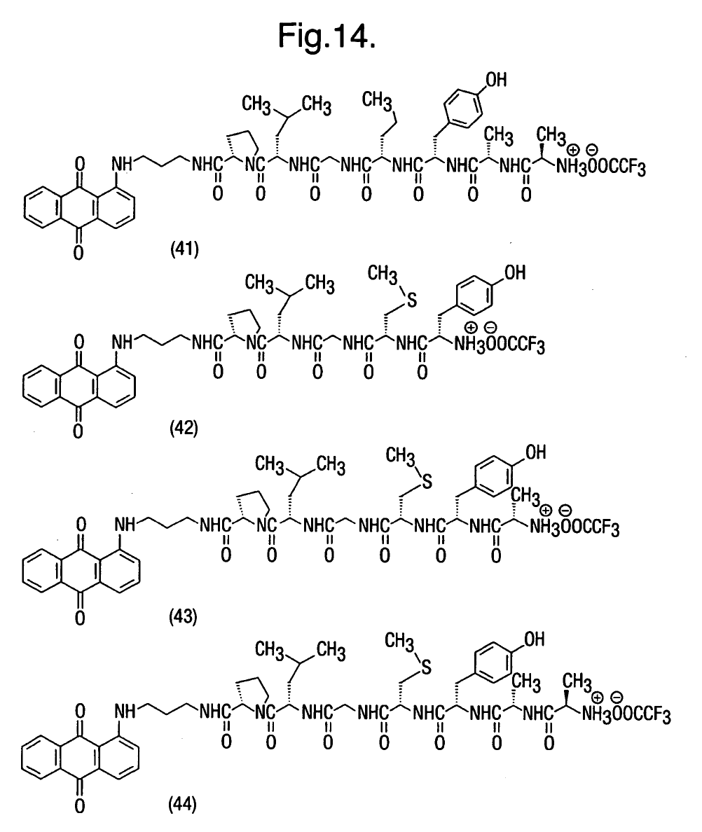

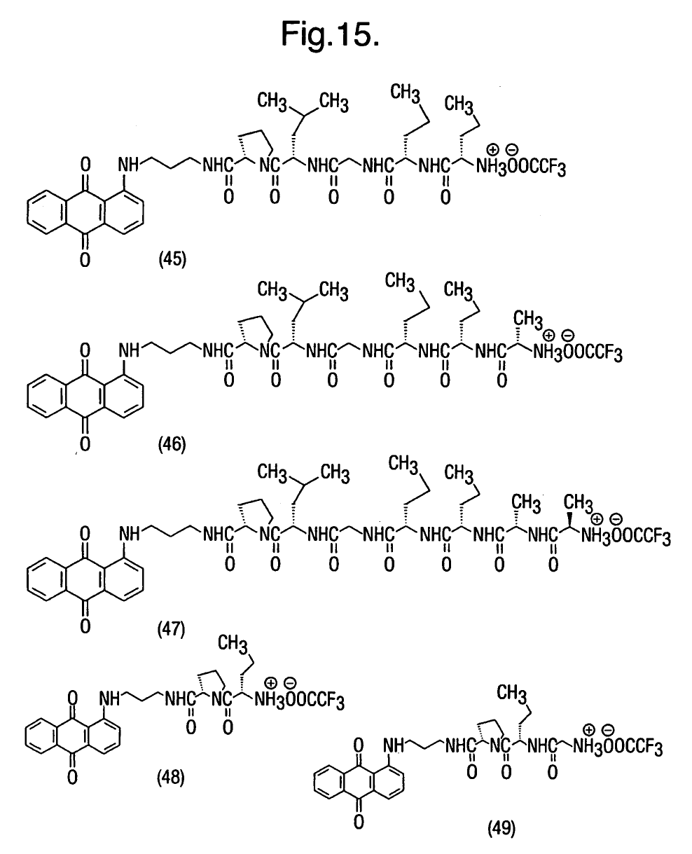

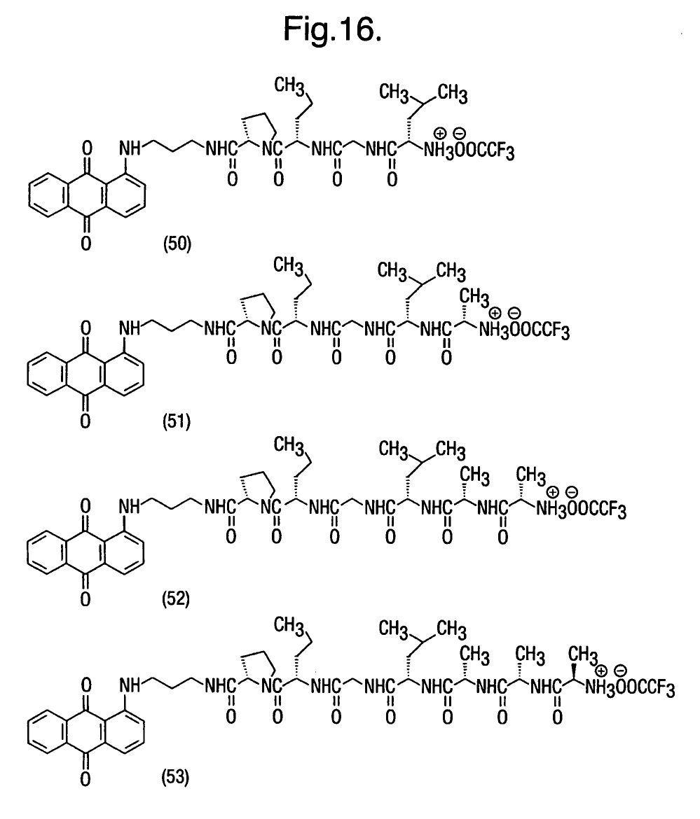

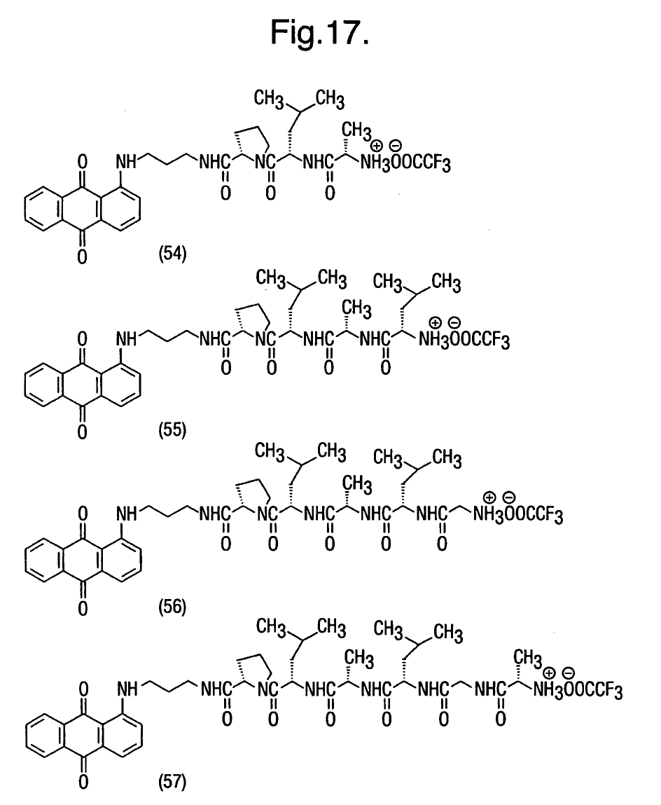

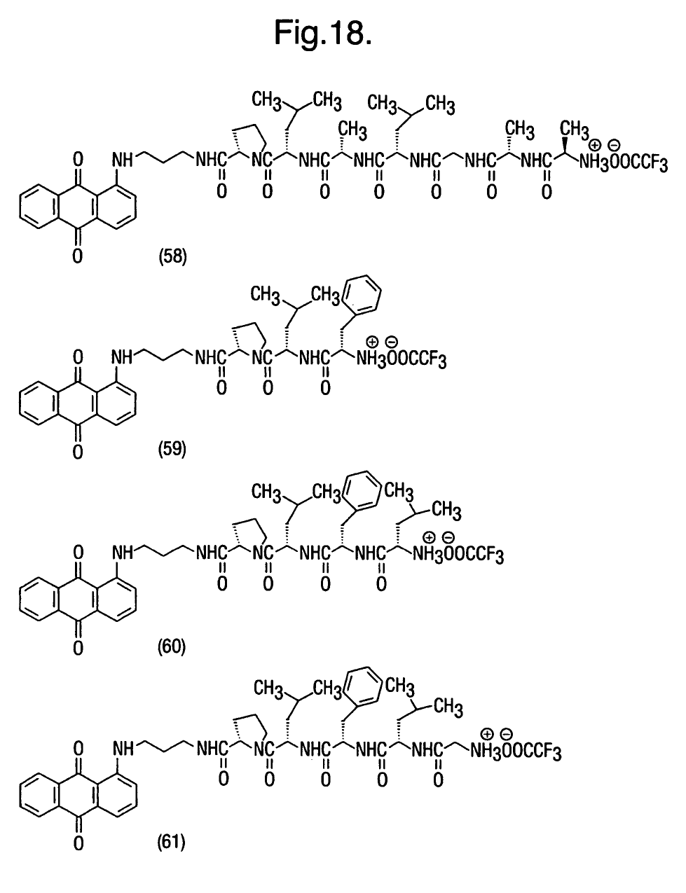

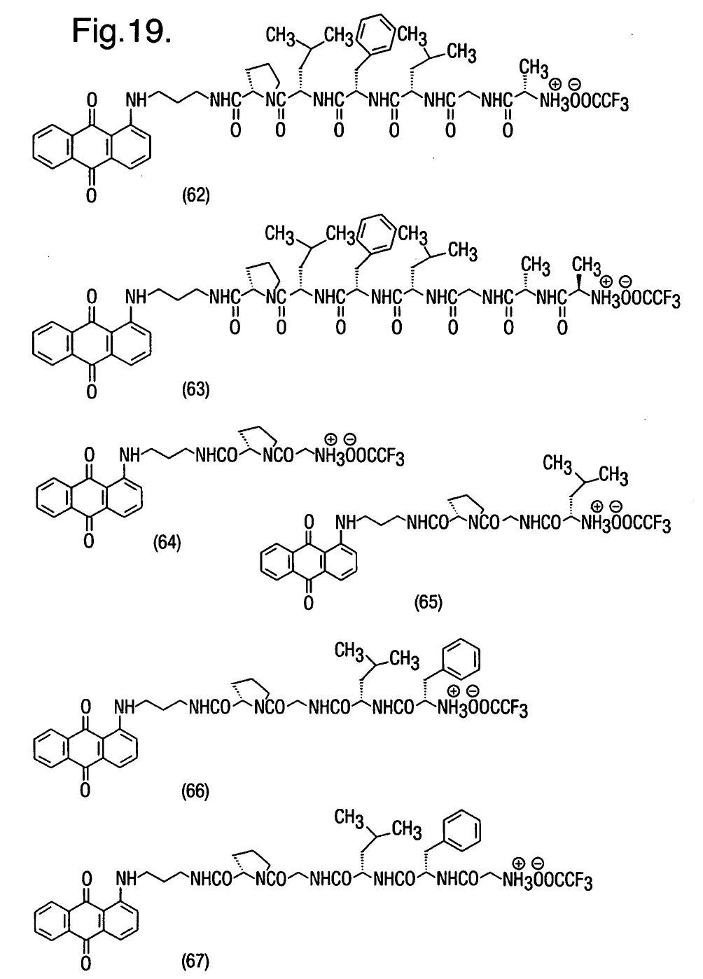

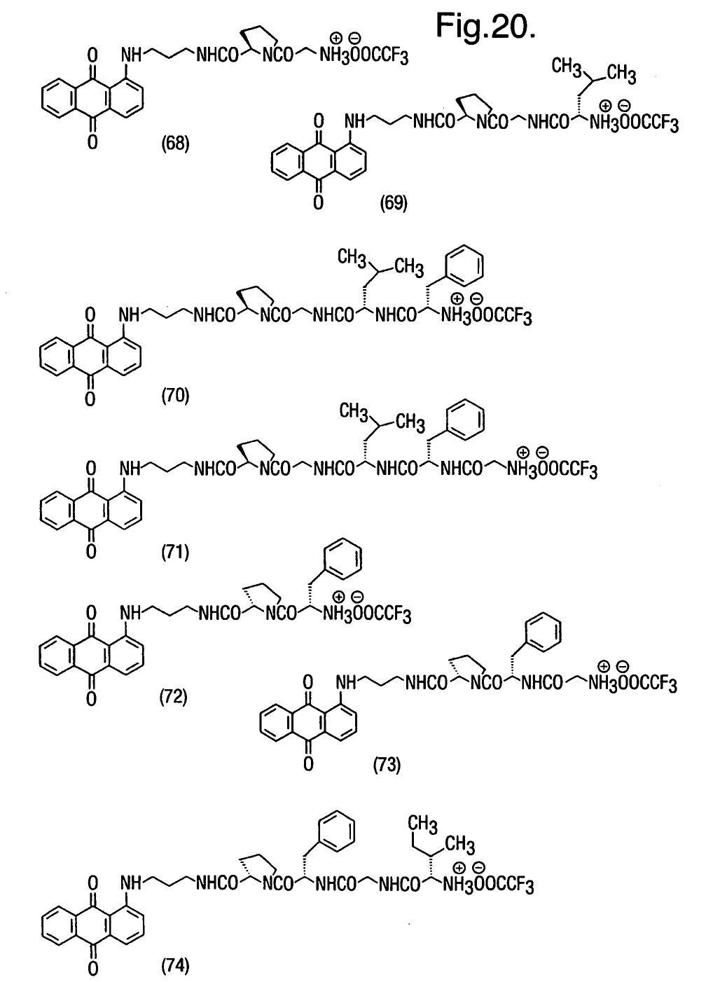

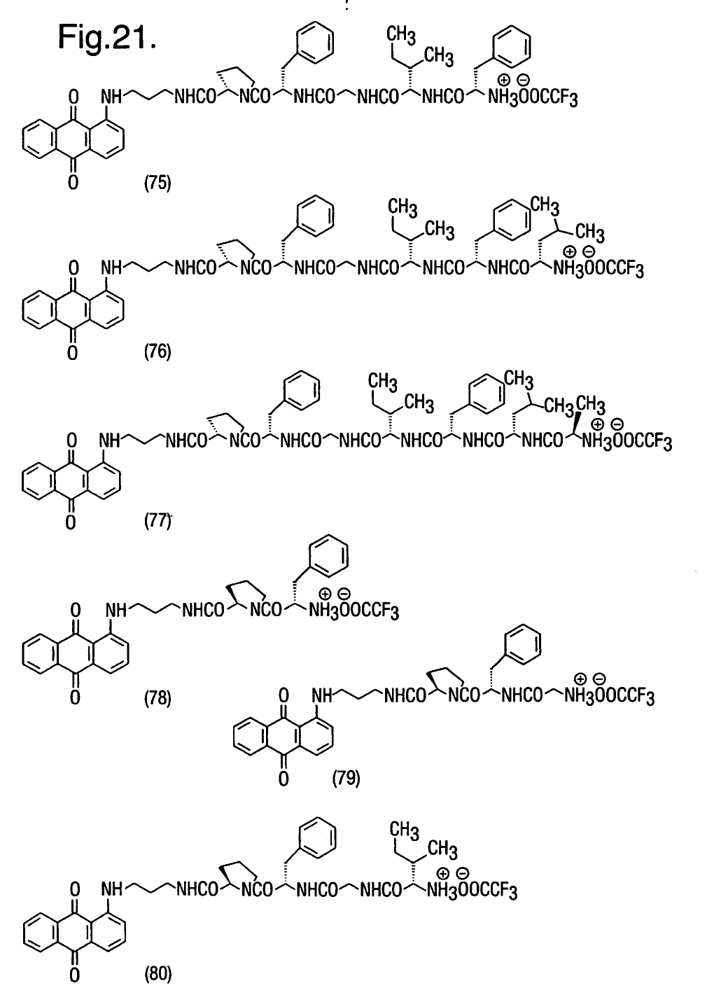

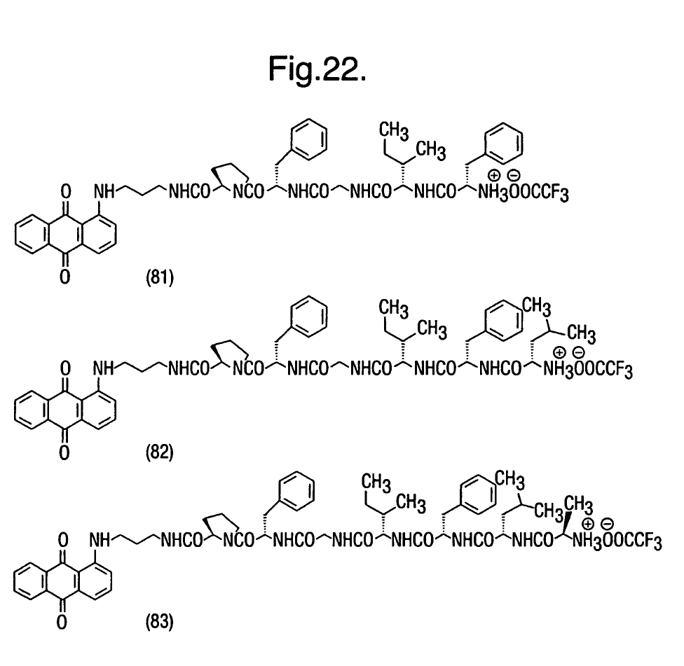

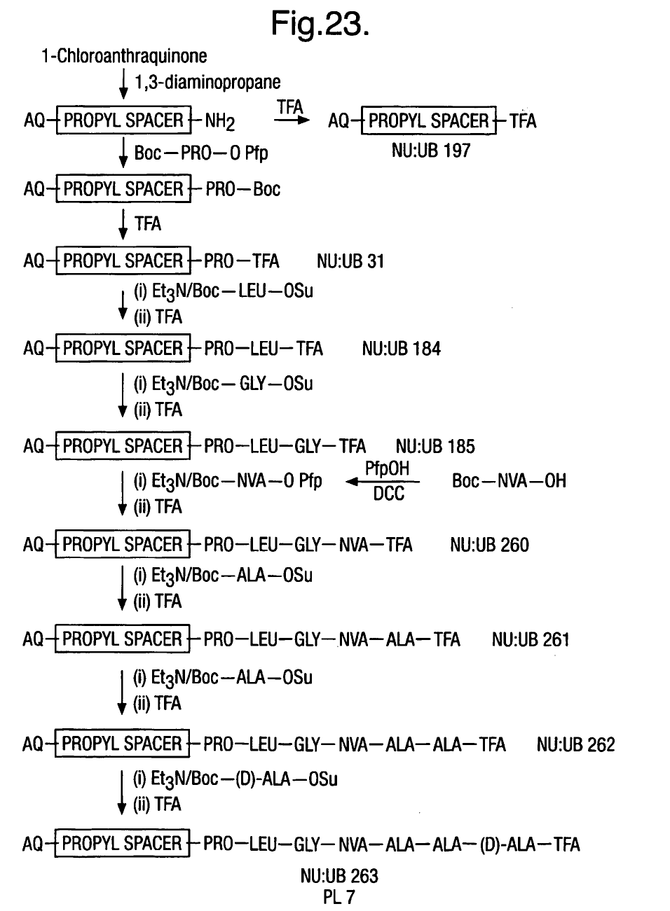

Example 8 and intermediate MMP cleavage product metabolites were characterised by combined hplc/mass spectrometric analysis and MS/MS (EPSRC Centre, Swansea) generating comprehensive and characteristic sets of parent and product ion data, to define optimal substrates (FIGS. 2 and 3). Incubations of Example 8 with human recombinant MMP-9 (Calbiochem-Novabiochem Ltd) have shown quite clearly by both HPLC and MS/MS cleavage to anthraquinonyl, spacer-linked tripeptide Example 4. Such incubations have been shown to contain the parent ion m/z 548 for Example 4, which appears to be further degraded to Example 3 (m/z 491). Further chromatographic studies with dilute tumour homogenates (1:500) have demonstrated he extremely rapid metabolism of Example 8 (1.75 μmol/min/g tissue) by a homogenate of HT1080 with the major products also being the cleavage product Example 4 and the anthraquinone Example 1a. These results have been conformed with MS/MS.

Neither example (8) [NU:UB187] (heptapeptide) nor its hexa-, penta-, or tetra-peptide metabolites {examples (7) [NU:UB 205], (6) [NU:UB 204] and (5) [NU:UB 186] respectively} inhibited the topo I- mediated relaxation of pBR322 DNA at concentrations up to 100 μM. The principal MMP-9 cleavage product example (4) [NU:UB 185] weakly inhibited relaxation at 50 μM. Greatest inhibitory activity was observed for example (3) [NU:UB 184] (the preferred MMP-2 cleavage product of example (8) [NU:UB187] The topo I activities of example (2) [NU:UB 31] and example (1b) [NU:UB 197] bad previously been determined [Table 1].

The DNA binding properties of Example 8 [NU:UB 187] have been compared to Example (2) [NU:UB 31]. The latter is known to bind to DNA by a mixed modal, part intercalative, part groove binding mechanism. Example 8 [NU:UB 187] is a weak groove binding agent in comparison to example (2) NU:UB 31 and was found not to intercalate into DNA [Table 3]. Taken together the foregoing results suggest that Example 8 [NU:UB 187] should have little interaction with native DNA and thus not induce genotoxicity. The lack of DNA binding and reduced or absent interaction with topoisomerase may be correlated to the absence of in vitro cytotoxicity [Table 2].

### Data for Example (15) [NU:UB 227]

Results

Further chromatographic studies with dilute tumour homogenates of HT1080 (1:500) have demonstrated the metabolism of example (15) (2.5 μmol/min/g tissue) is as rapid as that for example (8). The major metabolite being the expected cleavage product example (11) [NU:UB 224]. Details are given in FIG. 4.

Example (8) was shown to have a half-life of 1.46 h in murine whole blood and shown to be stable (t½>13 h) in saline and mouse plasma at 37° C.

TABLE 1
INHIBITION OF TOPO I-MEDIATED
RELAXATION pBR322 DNA

| | NU:UB | |
|---|---|---|
| EXAMPLE | CODE | LEVEL OF INHIBITION |
| (1b) | 197 | Complete inhibition of relaxation at 25 μM |
| (2) | 31 | Complete inhibition of relaxation at 25 μM |
| (3) | 184 | Complete inhibition of relaxation at 5 μM |
| (4) | 185 | Partial inhibition of relaxation at 50 μM |
| (5) | 186 | No inhibition of relaxation between 1-100 μM |
| (6) | 204 | No inhibition of relaxation between 1-100 μM |
| (7) | 205 | No inhibition of relaxation between 1-100 μM |
| (8) | 187 | No inhibition of relaxation between 1-100 μM |

TABLE 2
IN VITRO CYTOTOXICITY OF EXAMPLE (8)
[NU:UB 187] AND THE POTENTIAL CLEAVAGE
PRODUCTS (METABOLITES)

| | Cleavage Product | | | | | |
|---|---|---|---|---|---|---|
| | EXAMPLE | | | | | |
| | (1b) | (2) | (3) | (4) | (5) | (8) |
| | NU:UB | NU:UB | NU:UB | NU:UB | NU:UB | NU:UB |
| | 197 | 31 | 184 | 185 | 186 | 187 |
| Conc. (μM) | | | | | | |
| 0 | 100 | 100 | 100 | 100 | 100 | 100 |
| 1 | 90 | 100 | 80 | 91 | 89 | 100 |
| 10 | 100 | 30 | 58 | 55 | 100 | 100 |
| 100 | 5 | 0 | 1 | 0 | 0 | 95 |
| IC₅₀ (μM) | 33 | 5 | 14 | 13 | 30 | >>100 |

CELL LINE-MAC15A
CELL NUMBER-1 × 10⁴ cells/ml (kill shown as % cells remaining)
EXPOSURE TIME-1 hour
SOLVENT-DMSO (100 mM stock in DMSO)
DILUENT-RPMI media

TABLE 3
Fluorescence quenching Q₅₀ values for Ethidium
Bromide and Hoechst Dye 33258 displacement of bound
complexes with calf thymus DNA

| EXAMPLE | NU:UB CODE | QE₅₀ (μM) | QH₅₀ (μM) |
|---|---|---|---|
| (2) | NU:UB 31 | 2 | 0.6 |
| (8) | NU:UB 187 | 17 | 4 |

TABLE 4
IN VITRO CYTOTOXICITY OF PRODRUGS AND
POTENTIAL CLEAVAGE PRODUCTS (METABOLITES)
AGAINST MAC15A ADENOCARCINOMA
OF THE COLON (EXPOSURE TIME-96 hours)

| EXAMPLE | NU:UB CODE | IC₅₀ μM |
|---|---|---|
| 2 | 31 | 4.3 |
| 3 | 184 | 2 |
| 8 | 187 | 36 |
| 9 | 46 | 3.5 |
| 10 | 223 | 7 |
| 11 | 224 | 22 |
| 12 | 225 | 30 |
| 14 | 226 | 38 |
| 15 | 227 | 40 |
| 45 | 293 | 4 |
| 46 | 294 | 8 |
| 47 | 295 | 22 |
| 72 | 254 | 3 |
| 73 | 255 | 2.5 |
| 74 | 256 | 2.5 |
| 75 | 257 | 20 |
| 76 | 258 | 21 |
| 77 | 259 | 24 |

Protocols for Metabolic Analysis.

HPLC Protocol

Separation was achieved using gradient reversed phase HPLC On a Lichrospher (25×4 mm) column.

Mobile phase A consisted of 10% acetonitrile: 90% TFA (0.05%) and mobile phase B of 60% acetonitrile: 40% TFA (0.05%). Optimum detection was at the λmax of 248 nm using a flow rate of 1.2 ml/min. The gradient profile was from 60% A to 5% A over 25 min.

Sample preparation for HPLC was by simple protein precipitation. 3 vols. of methanol were added to 1 vol. of sample (typically 100 μl) which was centrifuged at 3000 g for 5 min. The supernatant was injected directly onto the HPLC column.

In vitro Enzyme Incubations

Compounds were incubated in vitro with purified recombinant enzyme (either MMP-9 or MMP-2) (Calbiochem, Nottingham, UK). Typically, compounds (drugs) were incubated at 5 μM concentrations using 10 μl of enzyme, 10μl of drug and 90 μl of buffer at 25° C. The buffer used consisted of 200 mM NaCl, 50 mM Tris, 5 mM CaCl₂, 20 μM ZnSO₄ and 0.05% Brij35 at pH 7.6.

At various time points 20 μl samples were taken and 60 μl of methanol added to precipitate proteins as described above and the metabolism of the compounds was analysed by HPLC (as above).

In Vitro Tumour Incubations

Solid tumour (HTT1080—excised from NCI-Nu mice under a Home Office Licence) was homogenised (1:4) in MMP buffer. Tumour homogenates were incubated at 25° C. and compounds (drugs) were added to the homogenate to give a final concentration of 10 μM or 100 μM. Samples were taken at timed intervals and prepared for HPLC analysis as described above.

Where metabolism was so rapid tumour homogenates required dilution (to 1:500) before analysis.

Stability Studies

Stability of the compounds in saline, plasma and tissue culture medium at 37° C. was investigated at 100 μg/ml using the protocols described above. In brief, samples were incubated at 37° C. and methanol precipitation used prior to HPLC analysis.

References

1 Berman J. Green M. Sugg E. Anderegg R. Millington D S. Norwood D L. McGeehan J. Wiseman J. Rapid optimization of enzyme substrates using defined substrate mixtures. Journal of Biological Chemistry. 267(3):1434-7, 1992

2. Bibby M C, Making the most of rodent tumour systems in cancer drug discovery. British Journal of Cancer 79(11-12): 1633-40, 1999.

3 Bickett D M, Green M D, Berman J, Dezube M, Howe A S, Brown P J, Roth J T and McGeehan G M, A high throughput fluorogenic substrate for interstitial collagenase (MMP-1) and gelatinase (MMP-9). Analytical Biochemistry 212(1): 58-64, 1993.

4. Bickett D M, Green M D, Wagner C, Roth J T, Berman J and McGeehan G M, A high throughput fluorogenic substrate for stromelysin (MMP-3). Annals of the New York Academy of Sciences 732: 351-5, 1994.

5. Birkedal-Hansen H, Moore W G, Bodden M K, Windsor L J, Birkedal-Hansen B, DeCarlo A and Engler J A, Matrix metalloproteinases: a review. Critical Reviews in Oral Biology & Medicine 4(2): 197-250, 1993.

6. Brown P D, Bloxidge R E, Stuart N S, Gatter K C and Carmichael J, Association between expression of activated 72-kilodalton gelatinase and tumor spread in non-small-cell lung carcinoma. Journal of the National Cancer Institute 85(7): 574-8, 1993.

7. Calabrese C R and Loadman P M, Analysis of the imidazoacridinone C1311 by high-performance liquid chromatography. Journal of Chromatography B 690(1-2): 275-281, 1997.

8. Calabrese C R, Loadman P M, Lim L S E, Bibby M C, Double J A, Brown J E and Lamb J H, In vivo metabolism of the antitumor imidazoacridinone C1311 in the mouse and in vitro comparison with humans. Drug Metabolism and Disposition 27(2): 240-245, 1999.

9. Chen E I, Kridel S J, Howard E W, Li W, Godzik A and Smith J W, A unique substrate recognition profile for matrix metalloproteinase-2. Journal of Biological Chemistry. 277: 4485-4491, 2002.

10. Double J A and Bibby M C, Therapeutic index: a vital component in selection of anticancer agents for clinical trial. Journal of the National Cancer Institute 81(13): 988-94, 1989.

11. Endo K, Maehara Y, Baba H, Yamamoto M, Tomisaki S, Wantanabe A, Kakeji Y and Sugimachi K, Elevated levels of serum and plasma metalloproteinases in patients with gastric cancer. Anticancer Research 17: 2253-2258, 1997.

12. Fridman R, Toth M, Pena D and Mobashery S, Activation of Progelatinase B (MMP-9) by Gelatinase A (MMP-2). Cancer Research 55: 2548-2555, 1995.

13. Jizasa T, Fujisawa T, Suzuki M, Motohashi S, Yasufuku K, Yasukawa T, Baba M and Shiba M, Elevated levels of circulating plasma matrix metalloproteinase 9 in non-small cell lung cancer patients. Clinical Cancer Research 5(1): 149-53, 1999.

14. Jabbar S A B, Twentyman P R and Watson J V, The MTT assay underestimates the growth inhibitory effects of interferons. British Journal of Cancer 60: 85-90, 1989.

15. Kleiner D E and Stetler-Stevenson W G, structural Biochemistry and activation of matrix metalloproteinases. Current Opinion in Cell Biology 5: 891, 1993.

16. Kleiner D E and Stetler-Stevenson W G, Matrix metalloproteinases and metastasis. Cancer Chemotherapy & Pharmacology 43(Suppl): S42-51, 1999.

17. S. J. Kridel, Chen E I, Kotra L P, Howard E W, Mobashery S and Smith J W, Substrate hydrolysis by matrix metalloproteinase-9. Journal of Biological Chemistry. 276: 20572-20578, 2001.

18. Lauer-Fields L J, Broder T, Sritharan, Chung L, Nagase H and Fields G B, Kinetic analysis of MMP activity using fluorogenic triple-helical substrates. Biochemistry. 40(19): 5795-5803, 200

19. Loadman P M, Phillips R M, Lim L E and Bibby M C, Pharmacological properties of a new aziridinylbenzoquinone RH1 (2,5-diaziridinyl-3-(hydroxymethyl)-6-methyl-1,4-benzoquinone) in mice. Biochemical Pharmacology 59, 229-234, 2000.

20. Loadman P M, Bibby M C, Double J A, Al-Shakhaa W M and Duncan R, Pharmacokinetics of PK1 and doxorubicin in experimental colon tumour models with differing responses to PK1. Clinical Cancer Research 5, 3682-3688, 1999.

21. MacDougall J R and Matrisian L M, Contributions of tumor and stromal matrix metalloproteinases to tumor progression, invasion and metastasis. Cancer & Metastasis Reviews 14(4): 351-62, 1995.

22. Marquisee M J and Kauer J C, Collagenase-sensitive peptidyl-nitrogen mustards as potential antitumour agents. Journal of Medicinal Chemistry 21(12): 1188-1194, 1978.

23. McGeehan G M, Bickett D M, Green M, Kassel D, Wiseman J S and Berman J, Characterization of the peptide substrate specificities of interstitial collagenase and 92-kDa gelatinase. Implications for substrate optimization. Journal of Biological Chemistry 269(52): 32814-20, 1994.

24. Monteagudo C, Merino M J, Sanjuan J, Liotta L A and Stetlerstevenson W G, Immunohistochemical distribution of type-iv collagenase in normal, benign, and malignant breast-tissue. American Journal of Pathology 136(3): 585-592, 1990.

25. Moore A J, Loadman P M, Devine D A and Bibby M C, Extraction of the Synthetic Lytic Peptide, Cecropin-B, From Biological Fluid and Analysis Using Reversed-Phase High Performance Liquid-Chromatography. Journal of Chromatography B-Biomedical Applications 672(2): 225-231, 1995.

26. Mucha A, Cuniasse P, Kannan R, Beau F, Yiotakis A, Basset P and Dive V, Membrane type-1 matrix metalloproteinase and stromelysin-3 cleave more efficiently synthetic substrates containing unusual amino acids in their P_{1′} positions. Journal of Biological Chemistry. 273(5): 2763-2768, 1998.

27. Nagase H, Fields C G and Fields G B, Design and characterization of a fluorogenic substrate selectively hydrolyzed by stromelysin 1 (matrix metalloproteinase-3). Journal of Biological Chemistry 269(33): 20952-7, 1994.

28. Nakajima M, Welch D R, Wynn D M, Tsuruo T and Nicolson G L, Serum and plasma M(r) 92,000 progelatinase levels correlate with spontaneous metastasis of rat 13762NF mammary adenocarcinoma. Cancer Research 53(23): 5802-7, 1993.

29. Ponton A, Coulombe B and Skup D, Decreased expression of tissue inhibitor of metalloproteinases in metastatic tumor cells leading to increased levels of collagenase activity. Cancer Research 51(8): 2138-43, 1991.

30. Pyke C, Ralfkiaer E, Tryggvason K and Dano K, Messenger-RNA for 2 type-iv collagenases is located in stromal cells in human colon cancer. American Journal of Pathology 142(2): 359-365, 1993.

31. Rasmussen H S and McCann P P, Matrix metalloproteinase inhibition as a novel anticancer strategy: a review with special focus on batimastat and marimastat. Pharmacology & Therapeutics 75(1): 69-75, 1997.

32. Sato H, Kida Y, Mai M, Endo Y, Sasaki T, Tanaka J and Seiki M, Expression of genes encoding type IV collagen-degrading metalloproteinases and tissue inhibitors of metalloproteinases in various human tumor cells. Oncogene 7(1): 77-83, 1992.

33. Soyez H, Schacht E and Vanderkerken S, The crucial role of spacer groups in macromolecular prodrug design. Advanced Drug Delivery Reviews 21: 81-106, 1996.

34. Stack M S. Gray R D. Comparison of vertebrate collagenase and gelatinase using a new fluorogenic substrate peptide. Journal of Biological Chemistry. 264(8):4277-81, 1989

35. Stetler-Stevenson W G, Hewitt R and Corcoran M, Matrix metalloproteinases and tumor invasion: from correlation and causality to the clinic. Seminars in Cancer Biology 7(3): 147-54, 1996.

36. Steward W P, Marimastat (BB2516): current status of development. Cancer Chemotherapy & Pharmacology 43(Suppl): S56-60, 1999.

37. Swaine D J, Loadman P M, Bibby M C, Graham M A and Patterson L H, High-performance liquid chromatographic analysis of AQ4N-an alkylaminoanthraquinone N-oxide. Journal of Chromatography B: in press, 2000.

38. Timar F, Botyanszki J, Suli-Vargha H, Babo I, Olah J, Pogany G and Jeney A, The antiproliferative action of a melphalan hexapeptide with collagenase-cleavable site. Cancer Chemotherapy & Pharmacology 41(4): 292-8, 1998.

39. Tsuchiya Y, Sato H, Endo Y, Okada Y, Mai M, Sasaki T and Seiki M, Tissue inhibitor of metalloproteinase-1 is a negative regulator of the metastatic ability of a human gastric-cancer cell-line, kkls, in the chick-embryo. Cancer Research 53(6): 1397-1402, 1993.

40. Urbanski S J, Edwards D R, Maitland A, Leco K J, Watson A and Kossakowska A E, Expression of metalloproteinases and their inhibitors in primary pulmonary carcinomas. British Journal of Cancer 66(6): 1188-1194, 1992.

41. Wang X, Fu X, Brown P D, Crimmin M J and Hoffinan R M, Matrix metalloproteinase inhibitor BB-94 (batimastat) inhibits human colon-tumour growth and spread in a patient-like orthotopic model in nude-mice. Cancer Research 54(17): 4726-4728, 1994.

42. Westermarck J and Kahari V M, Regulation of matrix metalloproteinase expression in tumor invasion. FASEB Journal 13(8): 781-92, 1999.

1. A compound of general formula (I)
A-(B)ₙ—X (I)
wherein
A is a moiety comprising one or more of a heterocyclic ring, a carbocyclic ring and a fused ring system, the ring or ring system being essential for a biological activity of the compound by action at a nucleic acid or protein target
B is a bivalent spacer molecule attached directly to the ring system
n is an integer 0 or 1 and
X is a monovalent moiety containing an amide bond that is cleavable by the action of a matrix metalloproteinase enzyme such as to produce a compound of formula (II)
A-(B)ₙ-Q (II)
wherein Q is the moiety that would remain after the cleavage of X by the matrix metalloproteinase enzyme, and
wherein the biological activity of the compound of formula II is increased over that of the compound of formula (I).

2. A compound as claimed in claim 1, wherein the amide bond is a peptide bond between amino acids in a peptide chain X, which can be cleaved to result in a shorter peptide chain Q.

3. A compound as claimed in claim 1, wherein the amide bond is an amide bond between two moieties Xa and Xb, one or both of which include a peptide analogue.

4. A compound as claimed in claim 1, wherein X is (Xaa)ₘY and the compound of formula (I) is represented by a compound of formula (III)
A-(B)ₙ-(Xaa)ₘY (III)
wherein
Xaa is any amino acid residue
m is an integer from 2 to 100 and
Y is a hydrogen atom, a cation or a capping group,
wherein the ring or ring system is not directly attached to the α-carbon of an amino acid residue,
wherein Xaa is independently selected at each repeat occurrence such as to form an oligopeptide or protein, wherein the oligopeptide or protein is internally cleavable by a Matrix Metalloproteinase enzyme such as to produce a compound of formula (IV)
A-(B)ₙ-(Xaa)_{q}-Y (IV)
wherein q is 0 or an integer less than m, and
wherein the biological activity of the compound of formula (IV) is higher than that of the compound of formula (III).

5. A compound as claimed in claim 1, wherein the activity of the compound of formula (II) is one of modulation of the activity of a cell membrane or intracellular receptor, an enzyme, or modulation of the ability of DNA to be replicated, transcribed or expressed.

6. A compound as claimed in claim 3, wherein the compound of formula (III) has higher biological activity at the nucleic acid or protein target than a compound of formula (V).
A-(B)ₙ—Y (V).

7. A compound as claimed in claim 1, wherein the moiety A is selected from the group consisting of moieties comprising a fused ring system and moieties comprising an unfused heterocyclic ring.

8. A compound as claimed in claim 1, wherein moiety A is selected from the group consisting of moieties comprising a steroid ring system, an anthracene ring system and a mitomycin ring system, which are capable of interacting with a protein receptor or a nucleic acid.

9. A compound as claimed in claim 1, wherein the moiety A is selected from the group consisting of moieties comprising the rings of daunorubicin, mitoxanthrone, methotrexate, camptothecin, quinocarmycin, chlorambucil and mitomycin-C.

10. A compound as claimed in claim 4, wherein A is linked directly to the spacer B, or directly to (Xaa)ₘ, through a nitrogen atom.

11. A compound as claimed in claim 10, wherein the nitrogen atom is that of an amino group attached directly or indirectly to the ring.

12. A compound as claimed in claim 1, wherein the moiety B is selected from the group consisting of alkylene, α-ω-diamino, α-ω-dicarboxylate, α-ω-dialcohol, α-ω-aminoalcohol and α-ω-amino acid moieties.

13. A compound as claimed in claim 4, wherein the moiety B is of 2 to 10 contiguous atoms in length between A and the amino acid residue of (Xaa)ₘ to which B is linked.

14. A compound as claimed in claim 10, wherein the moiety B is of 4, 5 or 6 atoms long.

15. A compound as claimed in claim 14, wherein the moiety B is selected from the group consisting of —NH(CH₂)ₙ—C(O)—, —NH(CH₂)ₙ—NH—, and —NH(CH₂)ₙ—O—, wherein n is an integer from 2 to 4.

16. A compound as claimed in claim 4, wherein the oligopeptide chain (Xaa)ₘ is 4 to 30 amino acid residues long.

17. A compound as claimed in claim 16, wherein the oligopeptide chain is to 4 to 10 amino acid residues long.

18. A compound as claimed in claim 4, wherein the moiety (Xaa)_{q} is 1 to 8 amino acid residues long.

19. A compound as claimed in claim 18, wherein the moiety (Xaa)_{q} is 1 to 4 amino acid residues long.

20. A compound as claimed in claim 4, wherein the moiety (Xaa)ₘ is an oligopeptide chain of formula (VI):
-Xaa1-Xaa2-Xaa3-Xaa4- (VI)
wherein
Xaa1 is proline or a non-naturally occurring analogue thereof,
Xaa2 is leucine, a non-naturally occurring analogue thereof, S-mercaptoethyl cysteine (EMC), thienylalanine (THA) or p-chlorophenylalanine (PFC),
Xaa3 is glycine, a non-naturally occurring analogue thereof, thienylalanine (THA) or cyclohexylalanine (CHA), and
Xaa4 is leucine, a non-naturally occurring analogue thereof, S-methylcysteine (SMC), norvaline (NVA), norleucine (NLE) or phenylglycine (PHG).

21. A compound as claimed in claim 20, wherein the oligopeptide chain -(Xaa)ₘ is of formula (VII):
-Xaa1-Xaa2-Xaa3-Xaa4-Xaa5- (VII)
wherein Xaa5 is the residue of any amino acid.

22. A compound as claimed in claim 21, wherein Xaa5 is selected from the group of amino acid residues consisting of L-or D-residues of any of the following amino acids: tyrosine, methionine, phenylglycine, isoleucine, leucine and norvaline, and non-naturally occurring analogues thereof.

23. A compound as claimed in claim 21, wherein the oligopeptide chain -(Xaa)ₘ- is of formula (VIII):
-Xaa1-Xaa2-Xaa3-Xaa4-Xaa5-Xaa6- (VIII)
wherein Xaa6 is the residue of any amino acid.

24. A compound as claimed in claim 23, wherein Xaa5 is the residue of D or L-alanine or a non-naturally occurring analogue thereof.

25. A compound as claimed in claim 20, wherein the oligopeptide chain -(Xaa)ₘ- is of formula (IX):
-Xaa1-Xaa2-Xaa3-Xaa4-Xaa5-Xaa6-Xaa7- (IX)
wherein Xaa7 is L- or D-residue of any of the following amino acids: alanine, lysine or ornithine (ORN).

26. A compound as claimed in claimed 25, wherein Xaa7 is a D-amino acid residue.

27. A compound as claimed in claim 4, wherein the capping group Y is selected from the group consisting of a hydrogen atom, OH, a cation and a pharmaceutically acceptable capping group of a peptide chain.

28. A compound as claimed in claim 1, wherein the action of one or more specific matrix metalloproteinases on said compound increases the cytotoxicity of the product of said action to tumour cells.

29. A compound as claimed in claim 28, wherein the matrix metalloproteinase is selected from MMP-2 and MMP-9.

30. A compound as claimed in claim 2, comprising an anthraquinone ring system linked to the peptide through a bivalent spacer group.

31. A pharmaceutical preparation comprising a pharmaceutically acceptable carrier and/or excipient and a compound as claimed in claim 1.

32. A method of treating a disorder wherein the tissue affected produces matrix metalloproteinase (MMP), comprising administering to a human or animal body in need of the treatment an effective therapeutic dose of a compound as claimed in claim 1.

33. A method as claimed in claim 32 wherein the disorder is selected from the group consisting of cancer, viral infection, parasitic infection and inflammation.

34. (Cancelled)

35. (Cancelled)

36. A method for modulating the biological activity of a biologically active molecule, comprising conjugating a moiety of formula (X)
—(B)ₙ—X (X)
wherein
B is a bivalent spacer molecule
n is an integer 0 or 1
and X is a monovalent moiety containing an amide bond that is cleavable by the action of a matrix metalloproteinase enzyme such as to produce a compound of formula (XI)
—(B)ₙ-Q (XI)
wherein Q is the moiety that would remain after the cleavage of X by the matrix metalloproteinase enzyme,
with said biologically active molecule, wherein that molecule is active at a protein or nucleic action target,
wherein the conjugated molecule is made more active in the presence of matrix metalloproteinase than when the matrix metalloproteinase is absent.

37. A method according to claim 36, wherein —(B)ₙ—X is an oligopeptide comprising a sequence of general formula (XII):
-Xaa1-Xaa2-Xaa3-Xaa4- (XII)
wherein
Xaa1 is proline or a non-naturally occurring analogue thereof,
Xaa2 is leucine, a non-naturally occurring analogue thereof, S-mercaptoethyl cysteine (EMC), thienylalanine (THA) or p-chlorophenylalanine (PFC)
Xaa3 is glycine, a non-naturally occurring analogue thereof, thienylalanine (THA) or cyclohexylalanine (CHA), and
Xaa4 is leucine, a non-naturally occurring analogue thereof, S-methylcysteine (SMC), norvaline (NVA), norleucine (NLE) or phenylglycine (PHG).

38. A method according to claim 37, wherein the oligopeptide is of formula (XIII):
-Xaa1-Xaa2-Xaa3-Xaa4-Xaa5- (XIII)
wherein Xaa5 is the residue of any amino acid.

39. A method according to claim 38, wherein the oligopeptide is of formula (VIII)
-Xaa1-Xaa2-Xaa3-Xaa4-Xaa5-Xaa6- (VIII)
wherein Xaa6 is the residue of any amino acid.

40. A compound as claimed in claim 3, wherein the peptide analogue is a peptide isostere.

41. A method according to claim 38, wherein Xaa5 is a tyrosine residue or a non-naturally occurring analogue thereof.

42. A method according to claim 38, wherein Xaa5 is selected from L- or D-amino acids tyrosine, methionine, phenyglycine, isoleucine, leucine and norvaline.

43. A method according to claim 39, wherein the oligopeptide is of formula (IX)
-Xaa1-Xaa2-Xaa3-Xaa4-Xaa5-Xaa6-Xaa7- (IX)
wherein Xaa7 is a L- or D-residue of any of the following amino acids: alanine, lysine or ornithine (ORN).
